(19) United States
(12) Patent Application Publication
(10) Pub. No.: US 2011/0033448 A1
(43) Pub. Date: Feb. 10, 2011

(54) Inhibitors of LL-37 Mediated Immune Reactivity to Self Nucleic Acids

(76) Inventors: Michel Gilliet, Pearland, TX (US); Roberto Lande, Houston, TX (US); Yong-Jun Liu, Pearland, TX (US)

Correspondence Address: Nielsen IP Law LLC, 1177 West Loop South, Suite 1600, Houston, TX, 77027, US

(21) Appl. No.: 11/957,959

(22) Filed: Dec. 17, 2007

Related U.S. Application Data
(60) Provisional application No. 60/870,375, filed on Dec. 15, 2006.

(51) Int. Cl. A61K 39/395; C07K 16/18; C07H 21/04; C08B 37/10; C12N 15/63; C12N 5/00; A61K 31/7088; A61K 31/727; A61K 31/74; C12Q 1/02; A61K 38/00; A61K 39/02; A61P 35/00; A61P 31/04; A61K 39/12; A61K 39/00; A61K 39/295; A61P 31/12; A61K 38/55

(52) U.S. Cl. 424/133.1; 530/387.3; 530/388.2; 536/23.1; 536/21; 435/320.1; 435/375; 424/130.1; 424/152.1; 514/44 R; 514/56; 530/387.1; 435/29; 514/1.1; 424/234.1; 424/204.1; 424/277.1; 424/201.1; 514/21.91

(57) ABSTRACT

Methods and compositions for treating disease are provided. More particularly, methods and compositions of inhibiting pathogenic interferon production are prevented, which may be useful in the treatment of various diseases. In other embodiments, therapeutic compounds and methods for the treatment of autoimmune diseases and chronic inflammatory diseases are provided. One such method is a method for inhibiting pathogenic interferon production or inhibiting activation of plasmacytoid dendritic cells or treating an autoimmune or chronic inflammatory disease, which comprises inhibiting one or more of LL-37 and hCAP18.

### CROSS-REFERENCE TO RELATED APPLICATIONS

This application claims the benefit of U.S. Provisional Application Ser. No. 60/870,375 filed Dec. 15, 2006, which is incorporated herein by reference.

### BACKGROUND

Plasmacytoid dendritic cell precursors (pDC) are key effectors in innate antiviral immunity due to their unique ability to secrete large amounts of type I interferons (IFNs) α/β in response to viral stimulation. pDCs are activated to produce type I IFNs through Toll-like receptors (TLR)7 and TLR9, which are endosomal receptors recognizing viral RNA and DNA, respectively. Type I IFNs (IFN-α, IFN-β, IFN-ω) are members of a cytokine family including several structurally related IFN-α proteins and a single IFN-β protein binding to the type I IFN surface receptor. Type I IFNs inhibit viral replication, increase the lytic potential of NK cells, increase expression of class I MHC molecules and stimulate the development of T helper 1 cells in humans. pDC are a rare cell population in the peripheral blood and secondary lymphoid organs characterized by plasma cell-like morphology and a unique surface phenotype. Virally exposed pDC subsequently differentiate into T cell stimulatory dendritic cells (DC) themselves or induce maturation of bystander myeloid DC through IFN-α, thus providing a unique link between innate and adaptive anti-viral immunity. During homeostasis, pDC are encountered exclusively in blood and lymphoid organs, however viral infection leads to active recruitment of pDC from the blood into peripheral sites of primary infection. pDC may also accumulate in peripheral tissues of certain noninfectious inflammatory disorders such as allergic contact dermatitis, cutaneous lupus erythematosus and psoriasis. A pathogenic role of pDC-derived type I IFNs in the induction of autoimmune inflammation has been shown in psoriasis (J Exp Med. 2005; 202(1):135-43), SLE (Science. 2001; 294(5546):1540-3), Sjogren's disease (Nat Clin Pract Rheumatol. 2006; 2(5):262-9), polymyositis (Ann Neurol. 2005; 57(5):664-78), rheumatoid arthritis (J Immunol. 2004; 173(4):2815-24), and proposed for type I diabetes mellitus (Clin Immunol. 2004; 111(3):225-33).

Self-non self discrimination can be explained by the invariant molecular nature of foreign ligands for innate receptors such as TLRs. This is particularly true for pathogen-derived ligands recognizing TLR expressed on the cell surface (TLR 1, 2, 4, 5, 6, 10). However structural differences between pathogen and host nucleic acids appear less prominent. Foreign versus self-discrimination is controlled by endosomal compartmentalization of the nucleic acid recognizing TLR. Pathogen-derived DNA may access TLR9 in the endosome of infected cells whereas host (self) DNA may not because rapidly degraded in the extracellular compartment by nucleases. Although during tissue damage as well as during the initiation and maintenance of autoimmune inflammation nucleic acids released by dying cells have been implicated in the initiation of the inflammatory process, it is unclear how this occurs.

The epithelial lining of the skin, gastrointestinal tract and bronchial tree produces a number of peptides with antimicrobial activities termed antimicrobial peptides (AMPs), which appear to be involved in both innate host defense and adaptive immune responses (Yang D. et al., 2001. Cell Mol Life Sci. 58:978-89). AMPS are cationic peptides which display antimicrobial activity at physiological concentrations under conditions prevailing in the tissues of origin. AMP synthesis and release is regulated by microbial signals, developmental and differentiation signals, cytokines and in some cases neuroendocrine signals, in a tissue-specific manner. Their mode of action is unknown, however the leading theory claims that permeabilization of target membranes is the crucial step in AMP-mediated antimicrobial activity and cytotoxicity. AMPS appear to have common characteristics that enable them to affect mammalian cells in a way that does not necessarily function through a ligand-receptor pathway, and that, being small, and highly ionic or hydrophobic or structurally amphiphilic, AMPS can bind mammalian cell membranes. They are able to penetrate through the cell membrane to the cytoplasm. For example, it was shown that granulysin penetrates and damages human cell membranes dependent upon negative charge (J. Immunol., 2001, 167:350-356). At high concentrations they are cytotoxic to cells; they tear through the membrane causing lysis or apoptosis.

Cathelicidins, one of the major classes of AMPS, contain a conserved “cathelin” precursor domain. Their organization includes an N-terminal signal peptide, a highly conserved prosequence, and a structurally variable cationic peptide at the C-terminus. The prosequence resembles cathelin, a protein originally isolated from porcine neutrophils as an inhibitor of cathepsin L (hence, the name cathelin). In humans there is only one cathelicidin named LL-37. The ability of cathelicidins, such as LL-37, to both kill bacteria and regulate immune responses is a characteristic of numerous AMPS. The peptide can influence host immune responses via a variety of cellular interactions, for example, it has been suggested to possibly function as a chemoattractant by binding to formyl-peptide-receptor-like-1 (FPRL-1). LL-37 can recruit mast cells, then be produced by the mast cell to kill bacteria.

LL-37 is a broadly expressed in a variety of cells, tissues and body fluids including, but not limited to, leukocytes, myelocytes, metamyelocytes, bone marrow, breast milk, skin of newborn infants, numerous squamous epithelia, nail, sweat, wound fluid, blister fluid, ocular surface epithelia, synovial membranes, nasal mucosa, lung epithelia, developing lung tissue, bronchoalveolar lavage fluid, salivary glands, saliva, gingiva, colon epithelium, colon mucosa, testis, epididymis epithelium, spermatozoa, seminal plasma, vernix caseosa, amniotic fluid, central nervous system (Biochimica et Biophysica Acta (BBA)—Biomembranes Volume 1758, Issue 9, September 2006, Pages 1408-1425). LL-37 plays a pivotal role in the response to tissue damage. LL-37 is rapidly and potently produced by epithelial cells (such as keratinocytes) upon injury (sterile or after microbial infection). Expression is terminated upon completed re-epithelialization. Furthermore LL-37 is constitutively expressed by granulocytes and released by degranulation after granulocyte infiltration of the damaged tissue.

LL-37 is upregulated in a number of disease states. In particular, LL-37 is highly expressed in keratinocytes of psoriasis and contact dermatitis. Furthermore LL-37 is highly expressed in inflamed synovial membranes, in gastric epithelia of Helicobacter pylori infections, in chronic nasal inflammatory disease, and has been described in the bronchoalveolar lavage of sarcoidosis and cystic fibrosis. In systemic lupus erythematosus (SLE) LL-37 is among the top upregulated genes in patient blood during active disease (J Exp Med. 2003 Mar. 17; 197(6):711-23). LL-37 expression is abundant in the lungs of cystic fibrosis patients (Eur Resp J 2007. 29:624-632), and may be involved in human arteriosclerosis (Arteriosclerosis, Thrombosis and Vascular Biology 2006. 26:1551-57).

### SUMMARY

The present disclosure provides a pathway specific to pDC cell activation by host (self) nucleic acids that may lead to production of pathogenic interferons. By blocking steps in the signaling pathway, pathogenic interferon production associated with certain autoimmune and chronic inflammatory diseases may be inhibited, thereby treating such diseases.

The methods of the present disclosure provide for specific blocking of LL-37 induced immune reactivity to self nucleic acids (self-DNA and self-RNA) leading to pathogenic type I IFNs. Type I IFNs are broadly expressed and of key importance in anti-viral immunity. Tumor immunoediting blocking of type I IFNs may potentially lead to serious adverse events. By the present disclosure, it is provided that LL-37, an upstream specific inducer of type I IFNs by pDC may be inhibited in order to block only pathogenic type I IFN release in autoimmune and chronic inflammatory disease, while leaving unaffected the type I IFN pathway elicited during infections.

The present disclosure also provides compositions and methods for TLR9 agonist CpG-mediated therapy. Such may be used in the prevention and therapy of infectious disease; enhancing vaccines, and directing adaptive immunity without vaccine. We have shown that LL-37 can enhance IFN-α production by CpG sequences. And CpG sequences are widely used as adjuvants for anti-microbial vaccines, anti-tumor vaccines, and to inhibit allergic diseases such as asthma. Accordingly, LL-37 may be used to enhance immunogenicity of CpG. LL-37 may also be used to enhance immunogenicity of anti-microbial vaccines that contain microbial nucleic acids (e.g., live, inactivated or killed microbes).

LL-37 may be targeted to tumors in which spontaneous apoptosis (and thus free DNA and RNA released in the extracellular environment) is a common feature, in order to induce inflammation and reverse immunosuppression. Tumor apoptosis is spontaneous. Therefore, intratumoral injection of LL-37 as well as systemic administration of LL-37 may target dying tumor cells in order to induce local formation of LL-37/nucleic acid complexes and induce protective anti-tumor inflammation.

The features and advantages of the present invention will be readily apparent to those skilled in the art upon a reading of the description of the embodiments that follows.

### DRAWINGS

Some specific example embodiments of the disclosure may be understood by referring, in part, to the following description and the accompanying drawings.

FIG. 1 shows identification of LL-37 as the key IFN-inducing factor in psoriasis. a: Reversed-phase HPLC chromatogram of psoriatic skin extracts. IFN-α produced by pDCs after stimulation with HPLC fractions (inserted bars). Arrow indicates fraction 26.

FIG. 2 shows Main IFN-α inducing HPLC fraction (fraction 26) was analyzed by ESI-MS. The integrated data of peptides with a mass ranging between 2 and 11 kDa revealed a species with a mass of 4,493, corresponding to the antimicrobial peptide LL-37. In the raw mass-spec data LL-37 was detected as 4-, 5- (insert), 6-, 7-, and 8-fold charged species. Upon nanospray-ESI-MS/MS analyses of LysC digests of fraction 26 a LysC-digest-ion at m/z 723.864 could be identified, which after collision-induced fragmentation gave the sequence DFLRNLVPRTES. This sequence is identical with the predicted carboxy-terminal sequence of LL-37.

FIG. 3 shows that LL-37 mediates IFN-inducing activity of fraction 26. IFN-α produced by pDCs after stimulation with fraction 26, LL-37 (3.9 μM) or R837 in the presence of anti-LL-37 (clone 8A8.2) or control antibodies (IgG2b). < indicates below detection limit of 12.5 pg/ml.

FIG. 4 shows LL-37 induces activation of pDC to produce IFN-α. (A) PDC (5×10⁴) were stimulated for 24 h with wild-type LL-37 (wt-LL-37, closed diamonds) or mutated LL-37 (mut-LL-37, closed squares) at the given concentrations. IFN-α production by pDC was measured by ELISA of the supernatants. One representative experiment out of 5 is shown. (B) Clump formation of pDC cultured with wt-LL-37 and mut-LL-37 as an indication of pDC-activation. (C) Production of IFN-α, IL-6 and TNF-α by pDC stimulated with LL-37 (10 μM), TLR-9 agonist CpG-B (CpG-2006, 1 μM) and TLR7-agonist imiquimod (R837, 10 μM).

FIG. 5 shows LL-37 is strongly expressed in the epidermis of psoriasis lesions but is also present in the dermis in the vicinity of a large numbers of pDC. (A) Real-time PCR for LL-37 normalized to GAPDH of total RNA derived from skin of healthy donors and lesional skin of patients with psoriasis, cutaneous lupus erythematosus, and prurigo nodularis. (B) Immunohistochemical staining of LL-37 (left panel) and pDC-marker BDCA-2 (right panel) in a psoriatic skin lesion.

FIG. 6 shows IFN-α induction by LL-37 is mediated by self-DNA through toll-like receptor 9 (TLR-9) stimulation. (A) pDC were stimulated with LL-37 (10 μM) in the presence of pertussis toxin (PTX) or KN62 to block the FPRL-1 and the P2X pathway respectively. Furthermore agonistic W peptide and ATP were used to stimulate these pathways on pDC. (B) pDC were stimulated with LL-37 (10 μM) in the presence of increasing concentrations of chloroquine to block the endosomal TLR pathway. (C) PDC were pre-treated DNase I, TLR-9 inhibitor (IRS, 4 μM) or ctrl ODN sequences for 30 min and followed by incubation with LL-37 (10 μM), CpG 2216 (CpGA, 1 μM) or imiquimod (R837, 10 μg/ml) for 24 h (A). The culture supernatants were analyzed for IFN-α production by ELISA. One representative experiment of three is shown.

FIG. 7 shows LL-37 targets human genomic DNA to pDC leading to IFN-α production. (A) PDC were stimulated for 24 h with LL-37 alone, purified human genomic DNA extracted from fetal human skin (huDNA, 3 μg/ml), alone or huDNA in the presence of LL-37 (10 μM) or mut-LL-37. The amount of IFN-α in the supernatants were measured by ELISA. One representative experiment of three is shown. (B) Fluorophore-labeled DNA alone or premixed with LL-37 (10 μM) for 30 min at room temperature was added to the pDC for 2 h. After removal of the incubation medium, the cells were extensively washed with ice-cold PBS, 2% HS, 0.5 mM EDTA to remove unspecific extracellular fluorophore. PDC were stained with anti-CD123 mAb (APC) and than analyzed by flow cytometry. Results are presented as % Alexa488-DNA positive cells. One representative experiment of 3 is shown.

FIG. 8 shows that anti-DNA antibodies mixed with purified human genomic DNA are not sufficient to activate pDC to produce type I IFNs unless LL-37 is present. (A) IFN-α secreted by purified pDC after overnight stimulation with purified genomic DNA (extracted from fetal human skin) alone, or pre-complexed with either LL-37 (50 μg/ml) or anti-dsDNA antibody (clone 11B6, 3 μg/ml), or LL-37 plus anti-dsDNA. (B) Flow cytometry detection of human DNA⁺ pDCs stimulated for 4 h with human DNA-Alexa 488 alone or complexed with LL-37 and/or anti-dsDNA.

FIG. 9 shows that LL-37 is present in circulating immune complexes of systemic lupus erythematosus (SLE). Total IgG were purified from SLE sera of patient 1 and 2 by HPLC using a protein G column. LL-37 content was measured by ELISA (left panel). Total SLE serum or purified IgG were used to stimulate pDCs with or without magnetic depletion of LL-37 using mouse anti-LL-37 Abs followed by anti-mouse magnetic beads (right panel).

FIG. 10 shows LL-37 forms a complex with human DNA. (A) Emission spectra of human genomic DNA intercalated with Ethimidium bromide in the presence of increasing doses of LL-37. (B) Size exclusion HPLC of LL-37 alone, mut-LL-37 alone or DNA premixed with LL-37 or mut-37. The large arrowhead shows the compacting of DNA, the small arrow shows DNA aggregates. Absorbance scales are different to accommodate the DNA peak.

FIG. 11 shows heparin inhibits the ability of LL-37 to induce IFN-α. Heparin (an anionic sugar) was preincubated for ½ h with LL-37 before stimulating pDC (thus associating with self-DNA released by dying cells in culture) or before adding genomic human DNA and subsequently stimulating pDC.

FIG. 12 shows LL-37/DNA complex enters the endosomal compartment of pDC. (A) Confocal microscopy of Texas-red LL-37/DNA complex in pDC at 30 minutes (left panel) and 4 hours (middle panel) of incubation. The Texas-red LL-37/DNA complex colocalizes with membrane structures stained by FM. (B) Colocalization of fluorchrome labeled LL-37 (red) with Fluorochrome labeled hu-DNA (green) in pDC.

FIG. 13 shows LL-37 induces extracellular protection from degradation, aggregate formation and retention in the early endosomes of DNA. (A) PDC were stimulated for 24 h with phosphothiorated (PS) or phosphodiesteric (PO) CpG-B sequences with or without LL-37. The amount of IFNα in the supernatants were measured by ELISA. (B) PDC were stimulated for 24 h with phosphothiorated (PS) or phosphodiesteric (PO) control ODN non-CpG sequences with or without LL-37. The amount of IFN-α in the supernatants were measured by ELISA. (C) PDC were stimulated for 24 h with aggregated CpG-A sequences, single stranded (ss) CpGA sequences (obtained after heat and flash cooling) or ssCpG-A sequences preincubated with LL-37. The amount of IFNα in the supernatants were measured by ELISA. (D) Confocal microscopy of pDC incubated for 2 h with Dextran (red), Lyso-tracker (blue) with either CpG-B alone (upper panels) or CpG-B complexed with LL-37 (lower panels).

FIG. 14 shows CpG motifs in both dsDNA and ssDNA sequences are required for induction of type I IFN by LL-37/DNA complex.

FIG. 15 shows that LL-37 complexed with non CpG-containing ODN is capable of inhibiting activation of pDC by type I IFN inducers, such as CpG-A.

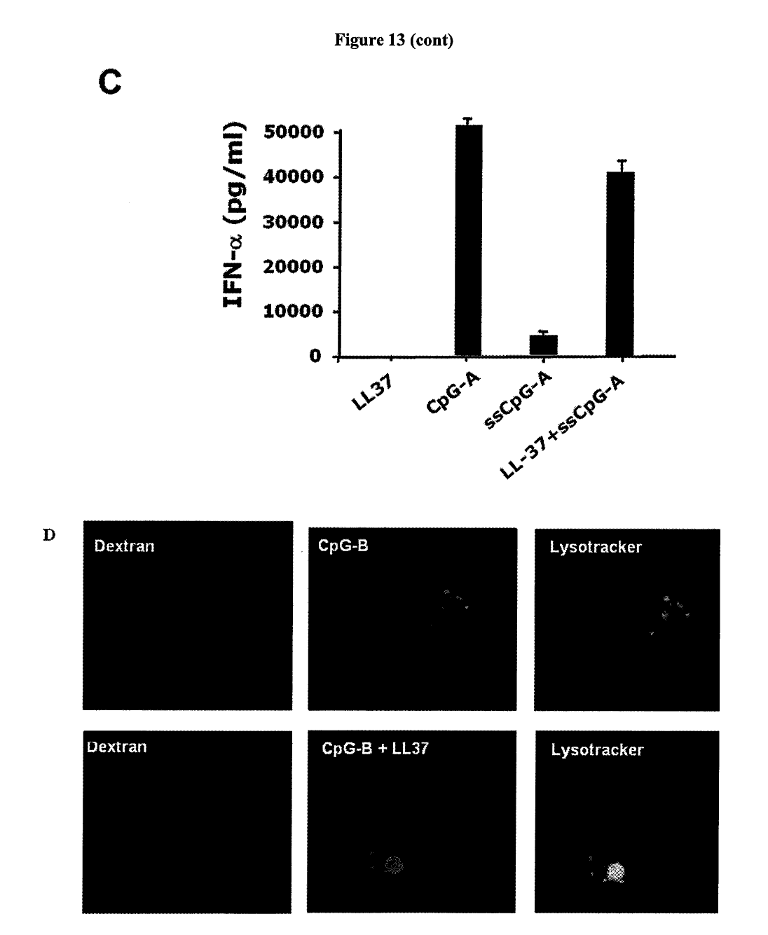

FIG. 16 shows human total RNA extracted from fetal skin can induce IFN-α in pDC when complexed with LL-37. RNA notably signals through endosomal TLR7 (expressed on pDC); it may also signal through endosomal TLR8 (expressed by myeloid dendritic cells not pDC) and thus may activate also other cell types than pDC.

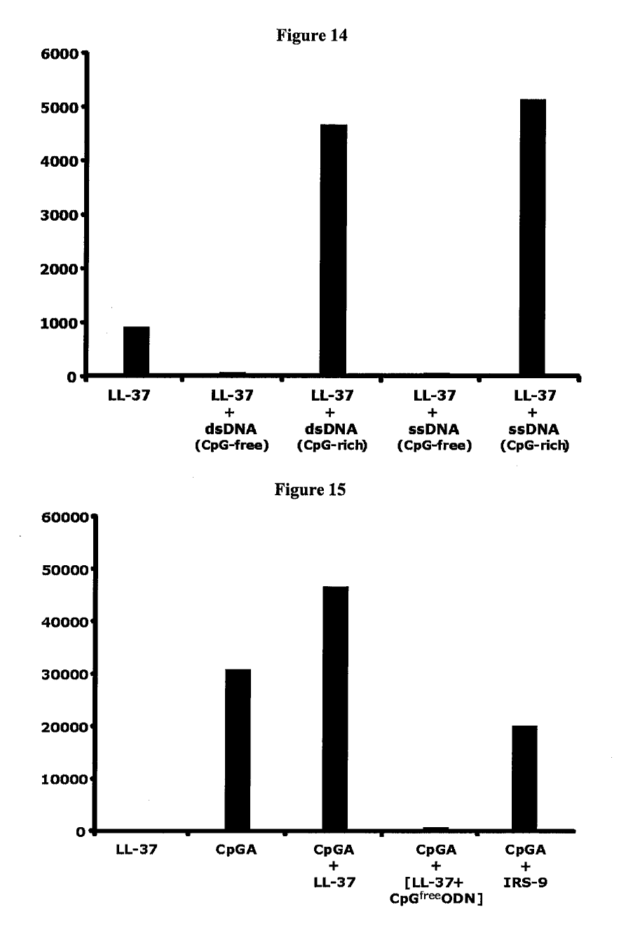

FIG. 17 shows that neutrophils release self-DNA-LL-37 complexes upon activation. (a) Human neutrophils purified from PBMC using anti-CD15 beads, were activated for 1 h with PMA or ionomycin and agarose gel electrophoresis was performed on cell-free supernatants with or without Dnase I treatment. (b) LL-37 in the supernatants of neutrophils activated as in (a) at different time-points measured by ELISA. (c) Confocal microscopy of purified, unstimulated neutrophils (left panel) or neutrophils stimulated for 2 h with PMA (right panel) stained with mouse anti-LL-37 (red) and YOYO-1 (green) to stain DNA.

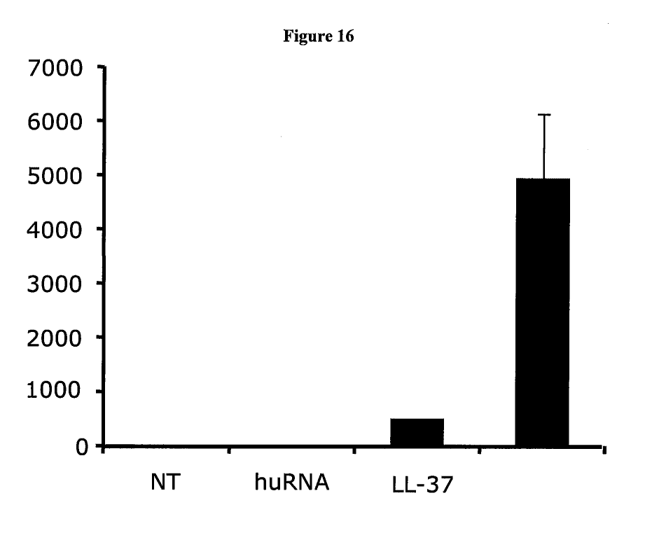

FIG. 18 shows that self-DNA-LL-37 complexes released by activated neutrophils activate pDC to produce type I IFNs. IFN-α produced by pDCs after stimulation for 24 h with either supernatant of activated neutrophils w/o DNase or LL-37 depletion (with anti-LL-37 Ab followed by beads-coated anti-mouse Abs).

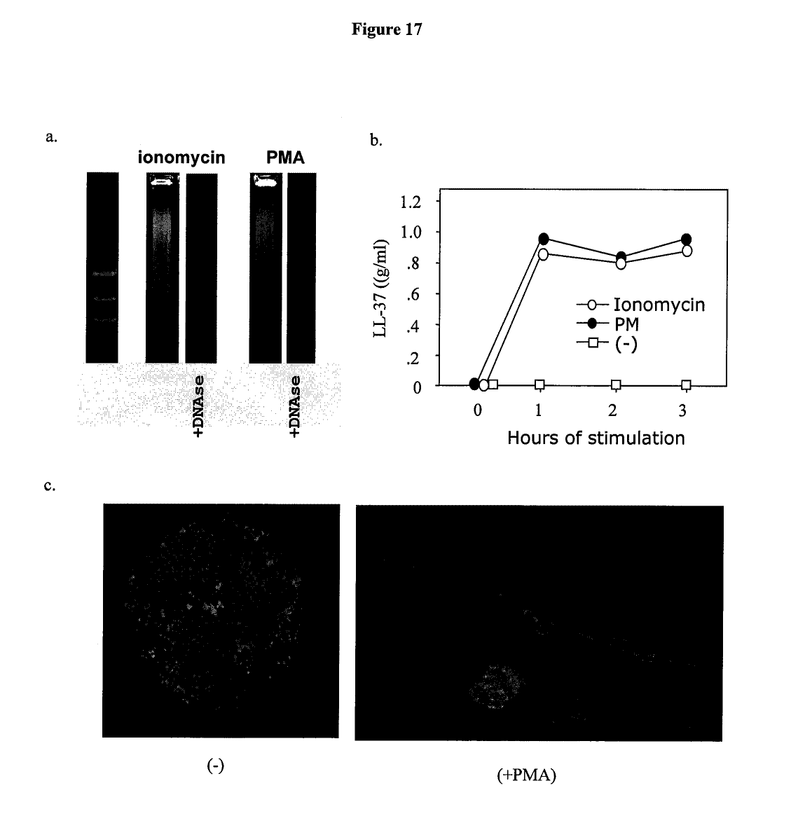

FIG. 19 shows that proteinase 3 inhibitors block the cleavage of LL-37 from its propeptide hCAP and inhibit the activation of pDC by self-DNA released by neutrophils. (Right panel) Western Blot of the sup from neutrophils activated with PMA (2 h) w/o pretreatment with Proteinase-3 inhibitors (P3i, Chymostatin and MeOSuc-CMK), or the same sup further treated with Proteinase-3 (P3), the serine-protease able to specifically cleave the peptide (LL-37, 4.5 kD) from the preprotein (hCAP18). (Left panel) IFN-α released by pDC stimulated with NET w/o Pr-3 inhibitors. CpG, w/o Pr-3 inhibitors is used as positive control.

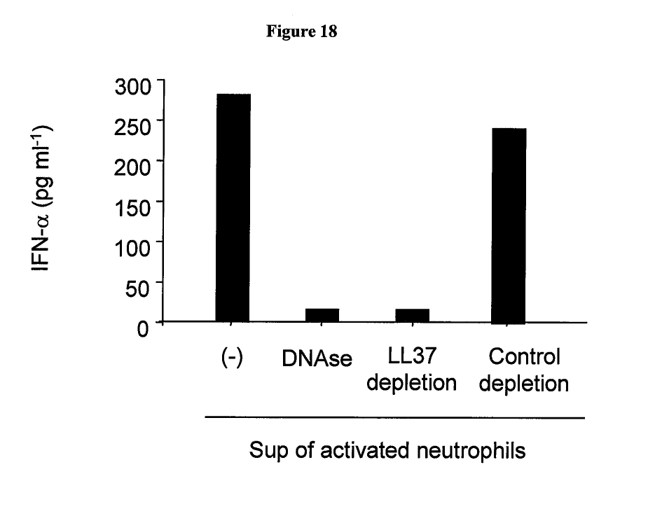

FIG. 20 shows that LL-37 converts genomic DNA of human and bacterial origin into potent IFN-α inducers. pDCs were stimulated with genomic DNA derived from human fetal skin, human lungs and human leukocytes (10 pg ml⁻¹) either alone or after premixing with LL-37 (10 μM). pDCs were also stimulated with genomic bacterial DNA isolated from Escherichia coli (E. coli) at 10 pg ml⁻¹. Levels of IFN-α were measured after overnight culture. <, indicates that the measured value was below the detection limit of the assay (12.5 pg ml⁻¹). Error bars represent the standard deviation of triplicate wells.

FIG. 21 shows that LL-37 converts self-RNA and viral RNA into activator of myeloid DC maturation and cytokine secretion. Myeloid (monocyte-derived) DC were stimulated with RNA isolated from U937 cells (human RNA) or a synthetic single-stranded RNA sequence derived from HIV (ssRNA40) and a known TL-7/8 ligand either alone (10 pg ml⁻¹) or after premixing with LL-37 (10 μM). (a) Maturation was assessed by flow cytometry analysis of CD80 after overnight culture. (b) Levels of TNF-α, IL-6, IL-12, and IL-23 were measured after overnight culture. <, indicates that the measured value was below the detection limit of the assay (12.5 pg ml⁻¹). Human DNA or CpG-DNA sequences did not activate mDC (not shown).

FIG. 22 shows that vaccination with LL-37 plus dying tumor cells induces prolonged survival of tumor challenged mice. 10⁶ A20 irradiated (5000 rad) were mixed with LL-37 (30 μg) or left in PBS alone and injected s,c. 7 days later mice were challenged with live A20 lymphoma i.v. 8 mice per group, survival over time is plotted.

FIG. 23 shows potent adjuvant activity of LL-37 for the induction of T cell mediated immunity. CD4+ T cells were purified from spleen and LN of HNT-TCR Tg mice (Thy 1.2), labeled with CFSE, and adoptively transferred (1×10⁶) into BALB/c Thy1.1 mice. Next day, mice were immunized s.c. with (a) 5×10⁶ A20 lysate plus HNT peptide and CpG-2216 (35 mg); (b) A20 lysate plus HNT peptide and LL-37 (35 mg); (c) A20F lysate plus HNT peptide; or (d) left untreated. Four days after immunization draining LN were harvested and Thy1.2 positive CD4 T cells were measured by flow cytometry.

FIG. 24 shows that intratumoral injection of LL-37 induces expression of pro-inflammatory and T-cell-derived cytokines. 100 mg of LL-37, CpG-A or PBS alone was injected into B16 tumors grown for 7 days in Flt-L treated mice. Tumors were harvested after 6, 24, 48 and 72 h, total RNA was extracted and expression of indicated cytokines was measured by real-time PCR. Data represent expression relative to GAPDH RNA. Some mice were injected with 100 mg of LL-37 for 3 times (t0, t24 and t48) and tumor was harvested at 72 h for RNA expression analysis.

FIG. 25 shows Melanoma metastases contain pDC and dying tumor cells but do not express LL-37. (a) Lineage⁻HLADR⁺CD123⁺ pDC in mononuclear cell suspensions of a subcutaneous melanoma metastasis. Tumor pDC coexist with dying 7-AAD⁺ tumor cells (b) Percentage of pDC among mononuclear cells in melanoma metastases in 4 independent specimen measured as in (a). (c) pDC identification by flow cytometry (left panel) and immunohistochemistry for BDCA-2. (d) LL-37mRNA expression relative to GAPDH mRNA in multiple melanoma metastases (n=19) specimen and psoriasis (n=12, positive control). < indicates <0.01.

FIG. 26 shows LL-37 binds and protects DNA released by dying tumor cells. (a) U937 were UV-irradiated to induce apoptosis and cultured for 24 h, or rendered necrotic by repeated freeze/thaw cycles and stained with Annexin V and PI to visualize apoptosis and necrosis. (b) 5×10⁶ live U937 cells (lines 1+2), or apoptotic UV-irradiated U937 (lines 3+4) were cultured for 24 h either alone or in the presence of LL-37 (50 mg/ml) before cell free supernatant was collected. U397 were also lyzed by freeze-thaw cycles to induce primary necrosis and cultured for 1 h either alone or in the presence of LL-37 (50 mg/ml) before cell free supernatant was collected. 20 ul of the supernatants in buffer were loaded onto 1% agarose gel and the electrophoresis was run for 1.5 hrs at 100V. The image was acquired with a Biorad gel imaging system.

FIG. 27 shows Murine pDC are activated by LL-37/DNA complexes to produce IFN-α in-vitro. Murine pDC were generated from Flt3 ligand supplemented BM cultures and isolated by sorting of CD11c+CD11b-B220+ cells, as previously described. 50,000 murine pDC in 20 ml of complete medium were stimulated with human LL-37 (10 mM), mouse CRAMP (30 mM), DNA alone, or DNA plus LL-37 or DNA plus CRAMP. After overnight culture supernatants were collected and tested for IFN-α by ELISA.

FIG. 28 shows Vaccination with LL-37 plus dying tumor cells induces prolonged survival of tumor challenged mice. 10⁶ irradiated A20 tumor cells were mixed with LL-37 (30 mg) or left in PBS alone and injected s,c. 7 days later mice were challenged intravenously with live 10⁷ A20 lymphoma cells. 8 mice per group, survival over time is plotted.

FIG. 29 shows single vaccination with LL-37 plus irradiated B 16 melanoma expressing OVA delays growth of pre-established B16-OVA skin tumor. Mice bearing a 7-d subcutaneous B16 melanoma transfected with a gene encoding OVA (B16-OVA)were vaccinated subcutaneously with 1) LL-37 alone; irradiated B16-OVA tumor (iB16-OVA); irradiated B16-OVA tumor mixed with 40 mg CpG-2216 (iB16-OVA+CpG); irradiated B16-OVA tumor with 40 mg LL-37 (iB16-OVA+LL-37). Tumor size was monitored by caliper every second day. Data represent mean of 4 mice per group.

FIG. 30 shows B16 melanoma contain large numbers of pDC. C57BL/6 mice were treated with the expression vector encoding a full-length murine Flt3 ligand cDNA, using the hydrodynamic-based gene delivery technique. After 4 days B16 tumor was implanted s.c. 7 days later, mice were sacrifized and tumor was analyzed. (left panel) Flow cytometry of tumor-derived single cell suspensions identifies large numbers of murine CD11c+B220+pDC in B16 tumors. (right panel) Immunohistochemistry for 3H3 (a specific marker for mouse pDCs) identifies pDC. pDCs were found in the vicinity of dying tumor cells as suggested by the large amounts of cell debris.

FIG. 31 shows Intratumoral injection of LL-37 induces early IFN-α expression. LL-37, CpG, or saline (PBS) was injected into B16 tumors grown for 7 days in Flt-L treated mice. Tumors were harvested after 6, 24, 48 and 72 h, total RNA was extracted and expression of indicated cytokines was measured by real-time PCR. Data represent expression relative to GAPDH RNA. Data is representative of 5 mice.

FIG. 32 shows LL-37 injection of tumors but not healthy muscle tissue induces type I IFN expression. 100 mg of LL-37 were injected into 7d-established B16 skin tumors and muscle tissue of the same mice. After 6 h tumor and muscle tissue were collected for RT-PCR analysis of IFN-a2 mRNA expression. Data represent expression relative to GAPDH RNA

FIG. 33 shows Single or repeated (3×) intratumoral injection of LL-37 delays growth of pre-established B16 tumor. Mice bearing a 7-d subcutaneous B16 melanoma were injected with 100 mg of LL-37 once (single), or repeatedly for 3 days (3×). Control injections were done with PBS. Tumor size was monitored by caliper every second day. Data represent mean of 4 mice per group.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure, according to specific example embodiments, generally relates to methods of treating disease. More particularly, the present disclosure relates to methods of inhibiting pathogenic interferon production. In other embodiments, the present disclosure provides therapeutic compounds and methods for the treatment of autoimmune diseases and chronic inflammatory diseases.

The present disclosure is based in part on the observation that pDC are key cells in infectious immunity due to their ability to produce large amounts of type I IFNs in response to microbial products. The aberrant activation of pDC is also critical for the initiation of autoimmune inflammation leading to disease formation. For example, it has been demonstrated that activation of pDC to produce type I IFNs occurs in the skin of patients with psoriasis and is an upstream event that initiates the local activation of autoimmune T cells and the development of skin lesions.

The present disclosure is further based in part on the observation that that LL-37, an endogenous antimicrobial peptide overexpressed in certain autoimmune diseases, can activate human pDC to produce type I IFNs. Targeting this pathway may provide effective treatment of autoimmune diseases in which the production of type I IFNs is escalated, such as, for example, psoriasis.

The present disclosure is further based in part on the discovery that self-DNA/RNA can become interferogenic if combined with LL-37. LL-37 is capable of forming complexes with endogenous human DNA/RNA in extracellular fluids and protects DNA/RNA from extracellular degradation. This complex is capable of efficiently targeting DNA/RNA to the endosomal compartment of pDC. This complex is endocytosed by pDC to trigger endosomal toll-like receptor 9/7 (TLR-9/7). Activation of this receptor leads to the production and secretion of type I IFNs.

Robust type I IFN production by pDC through endosomal TLR-9/7 has been recognized as being a central aspect of anti-viral immunity. Viruses infect pDC and enter the endosomal pathway to trigger TLR-9/7 through viral DNA/RNA. By contrast, human DNA/RNA released in the extracellular fluids by dying cells (either under homeostatic conditions or cell injury) fails to activate TLR-9/7 because it is rapidly degraded in the extracellular fluid and does not access the endosomal compartment. The expression of nucleic acid-specific TLR-9/7 in the endosomes but not on the cell surface represents a mechanism by which nature restricts the response to nucleic acids from invading microorganisms.

The present disclosure further provides a mechanism for the process by which sterile cell death with consequent release of endogenous DNA/RNA is linked to inflammation. As used herein, the term “sterile cell death” refers to cell death that occurs in the absence of microbes. This may occur if the DNA/RNA released by dying cells binds to LL-37. The complex will activate pDC to produce type I IFNs, a central pathway for the induction of inflammation. Although innate activation of pDC to produce type I IFNs has been recognized as key pathogenic event in a number of inflammatory conditions and autoimmune diseases, it has been unclear whether the activation signals were of microbial origin or whether endogenous ligands were involved. The present disclosure provides how inflammation occurs in non-infectious conditions, including, but not limited to autoimmune diseases and chronic inflammatory diseases.

The present disclosure further provides novel and specific therapeutic targets for the treatment of autoimmune disorders. The present disclosure further identifies targets for antagonistic monoclonal antibodies or molecular inhibitors (e.g., oligonucleotides) to affect the production of pathogenic interferons and to treat diseases associated with production of these interferons.

As used herein, the term “autoimmune disorder” refers to a disease caused by an inability of the immune system to distinguish foreign molecules from self molecules, and a loss of immunological tolerance to self antigens, that results in destruction of the self molecules. Autoimmune diseases, include but are not limited to, insulin-dependent diabetes mellitus (IDDM), diabetes mellitus, multiple sclerosis, experimental autoimmune encephalomyelitis (an animal model of multiple sclerosis), acute disseminated encephalomyelitis, rheumatoid arthritis, experimental autoimmune arthritis, myasthenia gravis, thyroiditis, an experimental form of uveoretinitis, Hashimoto's disease, primary myxoedema, thyrotoxicosis, pernicious anaemia, autoimmune atrophic gastritis, Addison's disease, premature menopause, male infertility, juvenile diabetes, Goodpasture's syndrome, pemphigus vulgaris, pemphigoid, sympathetic ophthalmia, phacogenic uveitis, autoimmune haemolytic anaemia, idiopathic leucopenia, primary biliary cirrhosis, active chronic hepatitis Hbₛ₋ᵥₑ, cryptogenic cirrhosis, ulcerative colitis, Sjogren's syndrome, scleroderma, Wegener's granulomatosis, Poly/Dermatomyositis, discoid LE, systemic Lupus erythematosus, Chron's disease, psoriasis, Ankylosing spondylitisis, Antiphospholipid antibody syndrome, Aplastic anemia, Autoimmune hepatitis, Coeliac disease, Graves' disease, Guillain-Barré syndrome (GBS), Idiopathic thrombocytopenic purpura, Opsoclonus myoclonus syndrome (OMS), Optic neuritis, Ord's thyroiditis, Pemphigus, Polyarthritis, Primary biliary cirrhosis, Rheumatoid arthritis, Reiter's syndrome, Takayasu's, Temporal arteritis, Warm autoimmune hemolytic anemia, Wegener's granulomatosis, Alopecia universalis, Behcet's disease, Chagas' disease, Chronic fatigue syndrome, Dysautonomia, Endometriosis, Hidradenitis suppurativa, Interstitial cystitis, Neuromyotonia, Sarcoidosis, Scleroderma, Ulcerative colitis, Vitiligo, and Vulvodynia.

The methods of the present disclosure may be used to treat any autoimmune or chronic inflammatory disease. In certain embodiments, the methods of the present disclosure may be useful to treat autoimmune diseases in which pDC-activation and type I IFN secretions have been shown to play a pathogenic role. Such diseases include, but are not limited to, psoriasis, systemic lupus erythematosus, Sjoegren's disease, polymyositis, diabetes mellitus type I, and multiple sclerosis. In other embodiments, the method of the present disclosure may be useful in treating autoimmune diseases characterized by increased expression of LL-37. Such diseases include, but are not limited to, inflammatory skin diseases, psoriasis, allergic contact dermatitis, H. pylory gastritis, chronic nasal inflammatory disease, cystic fibrosis, and sarcoidosis. In certain other embodiments, the methods of the present disclosure may be useful in treating postinfectious inflammatory disorders characterized by a self-sustaining cycle of tissue death and inflammation. In certain other embodiments, the methods of the present disclosure may be useful in treating graft versus host disease. In certain other embodiments, the methods of the present disclosure may be useful in treating arteriosclerosis, a disease in which LL-37 expression has been implicated.

The methods of the present disclosure may be used to inhibit a pathway, which results in the production of pathogenic interferons. For example, one such pathway that leads to the production of pathogenic interferons may involve LL-37 and TLR-9. LL-37, in humans, is cleaved extracellularly from an inactive propeptide, hCAP18. This cleavage results in formation of active LL-37. LL-37 is capable of binding endogenous DNA/RNA, thereby preventing DNA/RNA degradation. The binding of LL-37 and DNA creates a complex which interacts with the cell membrane of pDC, leading to endosomal uptake of the complex by pDC. This complex targets the endosomal compartment of pDC. Activation of pDC to produce type I IFNs by the LL-37/DNA complex is mediated by TLR-9, whereas the LL-37/RNA complex activates TLR-7. The complex is capable of activating nucleic acid-specific TLR-9/7, in the endosomes, which may cause production of type I IFNs. TLR-9/7 responses in pDC follow two pathways: an early endosomal response mediates by IRF7 with consequent induction of type I IFNs; and a late endosomal response mediated by NFkB and dominated by the induction of TNF-α, leading to maturation of the pDC into a dendritic cell.

The present disclosure provides compounds or molecules that inhibit the pathway leading to production of type I IFNs. Such compounds may include, but are not limited to, antibodies, oligonucleotides, and small molecules. The pathway may be inhibited at any of the steps described herein, which will lead to the inhibition of pDC activation and pathogenic IFN production.

In certain embodiments, production of LL-37 may be inhibited using oligonucleotide compounds (e.g., siRNA or antisense oligonucleotides). In these embodiments, oligonucleotides may be capable of specifically hybridizing with the mRNA transcript encoding for propeptide hCAP18.

In other embodiments, cleavage of LL-37 from propeptide hCAP18 may be prevented. In these embodiments, antibodies that bind the cleavage site of LL-37 may be generated using the peptide sequences spanning the cleavage site. Such techniques for antibody production are known in the art. Inhibition of cleavage of LL-37 from propeptide hCAP18 prevents the pathway leading the production of pathogenic IFNs through the LL-37/DNA complex.

In certain other embodiments, inhibiting or interfering with the binding of LL-37 to DNA may prevent activation of pDC and production of pathogenic IFNs. Activation of pDC to produce IFN-α by LL-37 is dependent on complex formation of LL-37 with DNA and the subsequent endosomal uptake of this complex by pDC. Accordingly, any molecule or compound capable of binding LL-37 will interfere with DNA binding, for example, monoclonal antibodies to LL-37. LL-37 further requires positive charges to form a complex with DNA, and any compound that is capable of neutralizing the positive charges of LL-37 will interfere with DNA binding as well. One such compound is a small molecule, such as heparin, may be used. A molecule or compound capable of binding LL-37 may also interfere with LL-37-pDC cell membrane interactions, which must occur prior to endosomal uptake of the complex by pDC. Prevention of endosomal uptake would thereby prevent pDC activation.

In certain other embodiments, TLR-9 and/or TLR-7 may be inhibited, which may block activity of the complex of LL-37 and DNA and/or RNA and may further prevent production of pathogenic IFNs. For example, a class of oligonucleotides, named immunoregulatory oligonucleotide sequences may be used to specifically bind and inhibit TLR-9 and/or TLR-7.

TLR9 Agonist CpG-Mediated Therapy

TLR9 detects unmethylated CpG dinucleotides, which are relatively common in the genomes of most bacteria and DNA viruses, but also occur in vertebrate genomes. The endosomal localization of TLR9 allows efficient detection of invading viral nucleic acids, while preventing “accidental” stimulation by CpG motifs within self DNA. The two bases to the 5′ and 3′ sides of the CpG dinucleotide comprise a CpG motif, one of which is sufficient for immune stimulation through TLR9. Besides the hexamer CpG motif, the immune-stimulatory activity of an oligodeoxynucleotide (ODN) is determined by the number of CpG motifs it contains (usually two to four are optimal), the spacing of the CpG motifs (usually at least two intervening bases, preferably thymine residues, is optimal), the presence of poly-G sequences or other flanking sequences in the ODN (effect depends on ODN structure and backbone), and the ODN backbone (a nuclease-resistant phosphorothioate backbone is the most stable but gives relatively weaker induction of IFN secretion from pDC compared with native phosphodiester linkages in the CpG dinucleotide.

For therapeutic applications CpG ODN are typically synthesized with at least partially phosphorothioate-modified (PS-ODN) backbones to provide nuclease resistance and increased half-life, and generally produce a greater immune-stimulatory effect.

In certain embodiments, the present disclosure provides for the prevention and therapy of infectious disease with a synthetic TLR9 ligand. By way of explanation, and not of limitation, if the normal function of TLR9 is to stimulate protective immunity against intracellular pathogens, then it could be proposed that prophylactic or therapeutic treatment with a synthetic TLR9 ligand would provide protection against an intracellular infectious challenge and/or eliminate a chronic infection. Indeed, studies in mice have demonstrated that the innate immune defenses activated by CpG ODN given by injection, inhalation or even by oral administration can protect against a wide range of viral, bacterial and even some parasitic pathogens, including lethal challenge with Category A agents or surrogates such as Bacillus anthracis, vaccinia virus, Francisella tularensis, and Ebola virus.

In other embodiments, the present disclosure provides for enhancing vaccines with a synthetic TLR9 ligand. TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines in numerous animal models. Conjugation of a CpG ODN directly to an antigen can enhance antigen uptake and reduce antigen requirements, but cysteine residues in peptides or proteins can also form spontaneous disulphide bonds with the phosphorothioate linkage in ODN, resulting in enhanced CTL responses without the difficulties of a separate conjugation step.

In other embodiments, the present disclosure provides for directing adaptive immunity without a vaccine using a synthetic TLR9 ligand. Typically, induction of effective antigen-specific immune responses has required a vaccine. However, there are several therapeutic fields in which TLR9 activation has been applied to achieve a similar effect, but without a vaccine. For example, although allergy vaccines with CpG ODN typically provide rapid redirection of allergic responses, inhaled CpG ODN monotherapy given repeatedly can prevent or treat allergic airway responses not only in mouse models but also in primates. Potential mechanisms that have been proposed to explain the somewhat counterintuitive anti-inflammatory effect of TLR9 stimulation on pulmonary inflammation include the induction of a TH1-like cytokine milieu that suppresses the TH2 response, systemic expression of IL-10 or transforming growth factor (TGF), and pulmonary expression of indoleamine (2,3)-dioxygenase (IDO).

Antibodies Targeted to LL-37 and hCAP18

The present disclosure contemplates antibodies having a human constant region that binds to molecules, ligands, or receptors of the signaling pathway in pDC leading to production of IFNs. The antibodies contemplated by the present disclosure may be capable of inhibiting the production of pathogenic interferons and may aid in treating diseases relating to such production, such as certain autoimmune diseases (e.g., psoriasis) and chronic inflammatory diseases. These antibodies may comprise a complete antibody molecule, having full length heavy and light chains; a fragment thereof, such as a Fab, Fab′, (Fab′)₂, or Fv fragment; a single chain antibody fragment (e.g. a single chain Fv), a light chain or heavy chain monomer or dimer; multivalent monospecific antigen binding proteins comprising two, three, four or more antibodies or fragments thereof bound to each other by a connecting structure; or a fragment or analogue of any of these or any other molecule with the same or similar specificity. Polypeptides produced recombinantly or by chemical synthesis, and fragments or other derivatives, may be used as an immunogen to generate the antibodies that recognize these molecules, receptors, ligands, or portions thereof.

“Antibody” as used herein includes polypeptide molecules comprising heavy and/or light chains which have immunoreactive activity. Antibodies include immunoglobulins which are the product of B cells and variants thereof, as well as the T cell receptor (TcR) which is the product of T cells and variants thereof. An immunoglobulin is a protein comprising one or more polypeptides substantially encoded by the immunoglobulin kappa and lambda, alpha, gamma, delta, epsilon, and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Subclasses of heavy chains are also known. For example, IgG heavy chains in humans can be any of IgG1, IgG2, IgG3, and IgG4 subclasses. Immunoglobulins or antibodies can exist in monomeric or polymeric form, for example, IgM antibodies which exist in pentameric form and/or IgA antibodies which exist in monomeric, dimeric, or multimeric form.

A typical immunoglobulin structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively. The amino acids of an antibody may be naturally or nonnaturally occurring.

Antibodies that contain two combining sites are bivalent in that they have two complementarity or antigen recognition sites. A typical natural bivalent antibody is an IgG. Although vertebrate antibodies generally comprise two heavy chains and two light chains, heavy chain only antibodies are also known. See Muyldermans et al., Trends in Biochem. Sci. 26(4):230-235 (1991). Such antibodies are bivalent and are formed by the pairing of heavy chains. Antibodies may also be multivalent, as in the case of dimeric forms of IgA and the pentameric IgM molecule. Antibodies also include hybrid antibodies wherein the antibody chains are separately homologous with referenced mammalian antibody chains. One pair of heavy and light chain has a combining site specific to one antigen and the other pair of heavy and light chains has a combining site specific to a different antigen. Such antibodies are referred to as bispecific because they are able to bind two different antigens at the same time. Antibodies may also be univalent, such as, for example, in the case of Fab or Fab′ fragments.

Antibodies exist as full length intact antibodies or as a number of well-characterized fragments produced by digestion with various peptidases or chemicals. The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc and/or Fv fragments. The term “antigen-binding fragment” refers to a polypeptide fragment of an immunoglobulin or antibody that binds antigen or competes with intact antibody (i.e., with the intact antibody from which they were derived) for antigen binding (i.e., specific binding).

Thus, for example, pepsin digests an antibody below the disulfide linkages in the hinge region to produce F(ab)₂, a dimer of Fab which itself is a light chain joined to V_{H}-CH1 by a disulfide bond. F(ab)₂ may be reduced under mild conditions to break the disulfide linkage in the hinge region, thereby converting the F(ab)₂ dimer into a Fab′ monomer. The Fab′ monomer is essentially a Fab fragment with part of the hinge region (see, e.g., Fundamental Immunology (W. E. Paul, ed.), Raven Press, N.Y. (1993) for a more detailed description of other antibody fragments). As another example, partial digestion with papain can yield a monovalent Fab/c fragment. See M. J. Glennie et al., Nature 295:712-714 (1982). While various antibody fragments are defined in terms of the digestion of an intact antibody, one of skill in the art will appreciate that any of a variety of antibody fragments may be synthesized de novo either chemically or by utilizing recombinant DNA methodology. Thus, the term antibody as used herein also includes antibody fragments produced by the modification of whole antibodies, synthesized de novo, or obtained from recombinant DNA methodologies. One skilled in the art will recognize that there are circumstances in which it is advantageous to use antibody fragments rather than whole antibodies. For example, the smaller size of the antibody fragments allows for rapid clearance and may lead to improved access to a treatment site.

Binding fragments are produced by recombinant DNA techniques, or by enzymatic or chemical cleavage of intact immunoglobulins. Binding fragments include Fab, Fab′, F(ab′)₂, Fabc, Fv, single chains, and single-chain antibodies. Other than “bispecific” or “bifunctional” immunoglobulins or antibodies, an immunoglobulin or antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

Recombinant antibodies may be conventional full length antibodies, hybrid antibodies, heavy chain antibodies, antibody fragments known from proteolytic digestion, antibody fragments such as Fv or single chain Fv (scFv), single domain fragments such as V_{H} or V_{L}, diabodies, domain deleted antibodies, minibodies, and the like. An Fv antibody is about 50 kD in size and comprises the variable regions of the light and heavy chain. The light and heavy chains may be expressed in bacteria where they assemble into an Fv fragment. Alternatively, the two chains can be engineered to form an interchain disulfide bond to give a dsFv. A single chain Fv (scFv) is a single polypeptide comprising V_{H} and V_{L} sequence domains linked by an intervening linker sequence, such that when the polypeptide folds the resulting tertiary structure mimics the structure of the antigen binding site. See J. S. Huston et al., Proc. Nat. Acad. Sci. U.S.A. 85:5879-5883 (1988). One skilled in the art will recognize that depending on the particular expression method and/or antibody molecule desired, appropriate processing of the recombinant antibodies may be performed to obtain a desired reconstituted or reassembled antibody. See, e.g., Vallejo and Rinas, Microbial Cell Factories 3:11 (2004), available at www.microbialcellfactories.com/content/3/1/11.

Single domain antibodies are the smallest functional binding units of antibodies (approximately 13 kD in size), corresponding to the variable regions of either the heavy V_{H} or V_{L} chains. See U.S. Pat. No. 6,696,245, WO04/058821, WO04/003019, and WO03/002609. Single domain antibodies are well expressed in bacteria, yeast, and other lower eukaryotic expression systems. Domain deleted antibodies have a domain, such as CH2, deleted relative to the full length antibody. In many cases such domain deleted antibodies, particularly CH2 deleted antibodies, offer improved clearance relative to their full length counterparts. Diabodies are formed by the association of a first fusion protein comprising two V_{H} domains with a second fusion protein comprising two V_{L} domains. Diabodies, like full length antibodies, are bivalent and may be bispecific. Minibodies are fusion proteins comprising a V_{H}, V_{L}, or scFv linked to CH3, either directly or via an intervening IgG hinge. See T. Olafsen et al., Protein Eng. Des. Sel. 17:315-323 (2004). Minibodies, like domain deleted antibodies, are engineered to preserve the binding specificity of full-length antibodies but with improved clearance due to their smaller molecular weight.

The T cell receptor (TcR) is a disulfide linked heterodimer composed of two chains. The two chains are generally disulfide-bonded just outside the T cell plasma membrane in a short extended stretch of amino acids resembling the antibody hinge region. Each TcR chain is composed of one antibody-like variable domain and one constant domain. The full TcR has a molecular mass of about 95 kD, with the individual chains varying in size from 35 to 47 kD. Also encompassed within the meaning of TcR are portions of the receptor, such as, for example, the variable region, which can be produced as a soluble protein using methods well known in the art. For example, U.S. Pat. No. 6,080,840 and A. E. Slanetz and A. L. Bothwell, Eur. J. Immunol. 21:179-183 (1991) describe a soluble T cell receptor prepared by splicing the extracellular domains of a TcR to the glycosyl phosphatidylinositol (GPI) membrane anchor sequences of Thy-1. The molecule is expressed in the absence of CD3 on the cell surface, and can be cleaved from the membrane by treatment with phosphatidylinositol specific phospholipase C (PI-PLC). The soluble TcR also may be prepared by coupling the TcR variable domains to an antibody heavy chain CH2 or CH3 domain, essentially as described in U.S. Pat. No. 5,216,132 and G. S. Basi et al., J. Immunol. Methods 155:175-191 (1992), or as soluble TcR single chains, as described by E. V. Shusta et al., Nat. Biotechnol. 18:754-759 (2000) or P. D. Holler et al., Proc. Natl. Acad. Sci. U.S.A. 97:5387-5392 (2000). Certain embodiments of the invention use TcR “antibodies” as a soluble antibody. The combining site of the TcR can be identified by reference to CDR regions and other framework residues.

The combining site refers to the part of an antibody molecule that participates in antigen binding. The antigen binding site is formed by amino acid residues of the N-terminal variable (V) regions of the heavy (H) and light (L) chains. The antibody variable regions comprise three highly divergent stretches referred to as hypervariable regions or complementarity determining regions (CDRs), which are interposed between more conserved flanking stretches known as framework regions (FRs). The term “region” can refer to a part or portion of an antibody chain or antibody chain domain (e.g., a part or portion of a heavy or light chain or a part or portion of a constant or variable domain), as well as more discrete parts or portions of said chains or domains. For example, light and heavy chains or light and heavy chain variable domains include CDRs interspersed among FRs. The term complementarity determining region (CDR), as used herein, refers to amino acid sequences which together define the binding affinity and specificity of the natural Fv region of a native immunoglobulin binding site. The term framework region (FR), as used herein, refers to amino acid sequences interposed between CDRs. These portions of the antibody serve to hold the CDRs in appropriate orientation (allows for CDRs to bind antigen). The three hypervariable regions of a light chain (LCDR1, LCDR2, and LCDR3) and the three hypervariable regions of a heavy chain (HCDR1, HCDR2, and HCDR3) are disposed relative to each other in three dimensional space to form an antigen binding surface or pocket. In heavy-chain antibodies or V_{H} domains, the antigen binding site is formed by the three hypervariable regions of the heavy chains. In V_{L} domains, the antigen binding site is formed by the three hypervariable regions of the light chain.

The identity of the amino acid residues in a particular antibody that make up a combining site can be determined using methods well known in the art. For example, antibody CDRs may be identified as the hypervariable regions originally defined by Kabat et al. See E. A. Kabat et al., Sequences of Proteins of Immunological Interest, 5^{th} ed., Public Health Service, NIH, Washington D.C. (1992). The positions of the CDRs may also be identified as the structural loop structures originally described by Chothia and others. See, e.g., C. Chothia and A. M. Lesk, J. Mol. Biol. 196:901-917 (1987); C. Chothia et al., Nature 342:877-883 (1989); and A. Tramontano et al., J. Mol. Biol. 215:175-182 (1990). Other methods include the “AbM definition,” which is a compromise between Kabat and Chothia and is derived using Oxford Molecular's AbM antibody modeling software (now Accelrys), or the “contact definition” of CDRs set forth in R. M. MacCallum et al., J. Mol. Biol. 262:732-745 (1996). Table 1 identifies CDRs based upon various known definitions:

TABLE 1
CDR definitions

| CDR | Kabat | AbM | Chothia | Contact |
|---|---|---|---|---|
| L1 | L24-L34 | L24-L34 | L24-L34 | L30-L36 |
| L2 | L50-L56 | L50-L56 | L50-L56 | L46-L55 |
| L3 | L89-L97 | L89-L97 | L89-L97 | L89-L96 |
| H1 (Kabat) | H31-H35B | H26-H35B | H26-H32 . . . | H30-H35B |
| | | | H34 | |
| H1 (Chothia) | H31-H35 | H26-H35 | H26-H32 | H30-H35 |
| H2 | H50-H56 | H50-H58 | H52-H56 | H47-H58 |
| H3 | H95-H102 | H95-H102 | H95-H102 | H93-H101 |

General guidelines by which one may identify the CDRs in an antibody from sequence alone are as follows:

LCDR1:
Start—Approximately residue 24.
Residue before is always a Cys.
Residue after is always a Trp, typically followed by Tyr-Gln, but also followed by Leu-Gln, Phe-Gln, or Tyr-Leu.
Length is 10 to 17 residues.

LCDR2:
Start—16 residues after the end of L1.
Sequence before is generally Ile-Tyr, but also may be Val-Tyr, Ile-Lys, or Ile-Phe.
Length is generally 7 residues.

LCDR3:
Start—33 residues after end of L2.
Residue before is a Cys.
Sequence after is Phe-Gly-X-Gly.
Length is 7 to 11 residues.

HCDR1:
Start—approximately residue 26, four residues after a Cys under Chothia/AbM definitions; start is 5 residues later under Kabat definition.
Sequence before is Cys-X-X-X.
Residue after is a Trp, typically followed by Val, but also followed by Ile or Ala.
Length is 10 to 12 residues under AbM definition; Chothia definition excludes the last 4 residues.

HCDR2:
Start—15 residues after the end of Kabat/AbM definition of CDR-H1.
Sequence before is typically Leu-Glu-Trp-Ile-Gly, but a number of variations are possible.
Sequence after is Lys/Arg-Leu/Ile/Val/Phe/Thr/Ala-Thr/Ser/Ile/Ala.
Length is 16 to 19 residues under Kabat definition; AbM definition excludes the last 7 residues.

HCDR3:
Start—33 residues after end of CDR-H2 (two residues after a Cys).
Sequence before is Cys-X-X (typically Cys-Ala-Arg).
Sequence after is Trp-Gly-X-Gly.
Length is 3 to 25 residues.

The identity of the amino acid residues in a particular antibody that are outside the CDRs, but nonetheless make up part of the combining site by having a side chain that is part of the lining of the combining site (i.e., that is available to linkage through the combining site), can be determined using methods well known in the art, such as molecular modeling and X-ray crystallography. See, e.g., L. Riechmann et al., Nature 332:323-327 (1988).

Antibodies suitable for use herein may be obtained by conventional immunization, reactive immunization in vivo, or by reactive selection in vitro, such as with phage display. Antibodies may also be obtained by hybridoma or cell fusion methods or in vitro host cells expression system. Antibodies may be produced in humans or in other animal species. Antibodies from one species of animal may be modified to reflect another species of animal. For example, human chimeric antibodies are those in which at least one region of the antibody is from a human immunoglobulin. A human chimeric antibody is typically understood to have variable region amino acid sequences homologous to a non-human animal, e.g., a rodent, with the constant region having amino acid sequence homologous to a human immunoglobulin In contrast, a humanized antibody uses CDR sequences from a non-human antibody with most or all of the variable framework region sequence and all the constant region sequence from a human immunoglobulin. Chimeric and humanized antibodies may be prepared by methods well known in the art including CDR grafting approaches (see, e.g., N. Hardman et al., Int. J. Cancer 44:424-433 (1989); C. Queen et al., Proc. Natl. Acad. Sci. U.S.A. 86:10029-10033 (1989)), chain shuffling strategies (see, e.g., Rader et al., Proc. Natl. Acad. Sci. U.S.A. 95:8910-8915 (1998), genetic engineering molecular modeling strategies (see, e.g., M. A. Roguska et al., Proc. Natl. Acad. Sci. U.S.A. 91:969-973 (1994)), and the like.

The terms “humanized antibody,” as used herein, refers to an antibody that includes at least one humanized immunoglobulin or antibody chain (i.e., at least one humanized light or heavy chain) derived from a non-human parent antibody, typically murine, that retains or substantially retains the antigen-binding properties of the parent antibody but which is preferably less immunogenic in humans. The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and CDRs (e.g., at least one CDR) substantially from a nonhuman immunoglobulin or antibody, and further includes constant regions (e.g., at least one constant region or portion thereof, in the case of a light chain, and preferably three constant regions in the case of a heavy chain).

The term “constant region” (CR) as used herein, refers to the portion of the antibody molecule which confers effector functions. Typically non-human (e.g., murine), constant regions are substituted by human constant regions. The constant regions of the subject chimeric or humanized antibodies are typically derived from human immunoglobulins. The heavy chain constant region can be selected from any of the five isotypes: alpha, delta, epsilon, gamma, or mu. Further, heavy chains of various subclasses (such as the IgG subclasses of heavy chains) are responsible for different effector functions and thus, by choosing the desired heavy chain constant region, antibodies with desired effector function can be produced. Preferred constant regions are gamma 1 (IgG1), gamma 3 (IgG3) and gamma 4 (IgG4). More preferred is an Fc region of the gamma 1 (IgG1) isotype. The light chain constant region can be of the kappa or lambda type, preferably of the kappa type. In one embodiment the light chain constant region is the human kappa constant chain and the heavy constant chain is the human IgG1 constant chain.

An antibody can be humanized by any method, which is capable of replacing at least a portion of a CDR of a human antibody with a CDR derived from a nonhuman antibody. Methods for humanizing non-human antibodies have been described in the art, examples of which may be found in U.S. Pat. Nos. 5,225,539; 5,693,761; 5,821,337; and 5, 859,205; U.S. Pat. Pub. Nos. 2006/0205670 and 2006/0261480; Padlan et al., FASEB J. 9:133-9 (1995); Tamura et al., J. Immunol. 164:1432-41 (2000). Preferably, a humanized antibody has one or more amino acid residues introduced into it from a source which is non-human. These non-human amino acid residues are often referred to as “import” residues, which are typically taken from an “import” variable domain. Humanization can be essentially performed following the methods of Winter and colleagues (see, e.g., P. T. Jones et al., Nature 321:522-525 (1986); L. Riechmann et al., Nature 332:323-327 (1988); M. Verhoeyen et al., Science 239:1534-1536 (1988)) by substituting hypervariable region sequences for the corresponding sequences of a human antibody. Accordingly, such “humanized” antibodies are chimeric antibodies wherein substantially less than an intact human variable domain has been substituted by the corresponding sequence from a non-human species. In practice, humanized antibodies are typically human antibodies in which some hypervariable region residues and possibly some framework (FR) residues are substituted by residues from analogous sites in rodent antibodies.

The choice of human variable domains, both light and heavy, to be used in making humanized antibodies is very important to reduce antigenicity and human anti-mouse antibody (HAMA) response when the antibody is intended for human therapeutic use. According to the so-called “best-fit” method, the human variable domain utilized for humanization is selected from a library of known domains based on a high degree of homology with the rodent variable region of interest (M. J. Sims et al., J. Immunol., 151:2296-2308 (1993); M. Chothia and A. M. Lesk, J. Mol. Biol. 196:901-917 (1987)). Another method uses a framework region derived from the consensus sequence of all human antibodies of a particular subgroup of light or heavy chains. The same framework may be used for several different humanized antibodies (see, e.g., P. Carter et al., Proc. Natl. Acad. Sci. U.S.A. 89:4285-4289 (1992); L. G. Presta et al., J. Immunol., 151:2623-2632 (1993)).

Humanized antibodies of the present disclosure also can be produced in a host cell transfectoma using, for example, a combination of recombinant DNA techniques and gene transfection methods as is well known in the art (e.g., Morrison, S., Science 229:1202 (1985)).

For example, to express the antibodies, or antibody fragments thereof, DNAs encoding partial or full-length light and heavy chains, can be obtained by standard molecular biology techniques (e.g., PCR amplification, site directed mutagenesis) and can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{L} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the present disclosure carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel, Gene Expression Technology. Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, and the like. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)), and polyoma. Alternatively, nonviral regulatory sequences may be used, such as the ubiquitin promoter or β-globin promoter.

In addition to the antibody chain genes and regulatory sequences, the recombinant expression vectors of the present disclosure may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665; and 5,179,017). For example, typically the selectable marker gene confers resistance to drugs, such as G418, hygromycin, or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, for example, electroporation, calcium-phosphate precipitation, DEAE-dextran transfection, and the like.

As an alternative to humanization, human antibodies can be generated. For example, it is now possible to produce transgenic animals (e.g., mice) that are capable, upon immunization (or reactive immunization in the case of catalytic antibodies) of producing a full repertoire of human antibodies in the absence of endogenous immunoglobulin production. For example, it has been described that the homozygous deletion of the antibody heavy-chain joining region (J_{H}) gene in chimeric and germ-line immunoglobulin gene array into such germ-line mutant mice will result in the production of human antibodies upon antigen challenge. See, e.g., B. D. Cohen et al, Clin. Cancer Res. 11:2063-2073 (2005); J. L. Teeling et al., Blood 104:1793-1800 (2004); N. Lonberg et al., Nature 368:856-859 (1994); A. Jakobovits et al., Proc. Natl. Acad. Sci. U.S.A. 90:2551-2555 (1993); A. Jakobovits et al., Nature 362:255-258 (1993); M. Bruggemann et al., Year Immunol. 7:33-40 (1993); L. D. Taylor, et al. Nucleic Acids Res. 20:6287-6295 (1992); M. Bruggemann et al., Proc. Natl. Acad. Sci. U.S.A. 86:6709-6713 (1989)); and WO 97/17852.

Alternatively, phage display technology (see, e.g., J. McCafferty et al., Nature 348:552-553 (1990); H. J. de Haard et al., J Biol Chem 274, 18218-18230 (1999); and A. Kanppik et al., J Mol Biol, 296, 57-86 (2000)) can be used to produce human antibodies and antibody fragments in vitro using immunoglobulin variable domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, and is reviewed in, e.g., K. S. Johnson and D. J. Chiswell, Curr. Opin. Struct. Biol. 3:564-571 (1993). Several sources of V-gene segments can be used for phage display. A repertoire of V genes from unimmunized human donors can be constructed and antibodies to a diverse array of antigens (including self-antigens) can be isolated essentially following the techniques described by J. D. Marks et al., J. Mol. Biol. 222:581-597 (1991) or A. D. Griffiths et al., EMBO J. 12:725-734 (1993). See also U.S. Pat. Nos. 5,565,332 and 5,573,905; and L. S. Jespers et al., Biotechnology 12:899-903 (1994). As indicated above, human antibodies may also be generated by in vitro activated B cells. See, e.g., U.S. Pat. Nos. 5,567,610 and 5,229,275; and C. A. K. Borrebaeck et al., Proc. Natl. Acad. Sci. U.S.A. 85:3995-3999 (1988).

Amino acid sequence modification(s) of the antibodies described herein are contemplated. For example, it may be desirable to improve the binding affinity and/or other biological properties of the antibody. Amino acid sequence variants of an antibody are prepared by introducing appropriate nucleotide changes into the antibody nucleic acid, or by peptide synthesis. Such modifications include, for example, deletions from, insertions into, and/or substitutions of residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of an antibody molecule include the fusion to the N- or C-terminus of an anti-antibody to an enzyme or a polypeptide which increases the serum half-life of the antibody.

Another type of variant is an amino acid substitution variant. These variants have at least one amino acid residue in an antibody molecule replaced by a different residue. The sites of greatest interest for substitutional mutagenesis include the hypervariable regions, but FR alterations are also contemplated. Conservative substitutions are shown in Table 3 below under the heading of “preferred substitutions.” If such substitutions result in a change in biological activity, then more substantial changes, denominated “exemplary substitutions” as further described below in reference to amino acid classes, may be introduced and the products screened.

TABLE 3
Amino acid substitutions

| Original Residue | Exemplary Substitutions | Preferred Substitutions |
|---|---|---|
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Nle | Leu |
| Leu (L) | Nle; Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Nle | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: Nle, Met, Ala, Val, Leu, Ile;

(2) neutral hydrophilic: Cys, Ser, Thr;

(3) acidic: Asp, Glu;

(4) basic: Asn, Gln, His, Lys, Arg;

(5) residues that influence chain orientation: Gly, Pro; and

(6) aromatic: Trp, Tyr, Phe.

Non-conservative substitutions will entail exchanging a member of one of these classes for a member of another class.

Any cysteine residue not involved in maintaining the proper conformation of the antibody may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

One type of substitutional variant involves substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity). In order to identify candidate hypervariable region sites for modification, alanine scanning mutagenesis can be performed to identify hypervariable region residues contributing significantly to antigen binding. Once such variants are generated, the panel of variants is subjected to screening as described herein and antibodies with superior properties in one or more relevant assays may be selected for further development.

Another type of amino acid variant of the antibody alters the original glycosylation pattern of the antibody by deleting one or more carbohydrate moieties found in the antibody and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences Asn-X″-Ser and Asn-X″-Thr, where X″ is any amino acid except proline, are generally the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of or substitution by one or more serine or threonine residues to the sequence of the original antibody (for O-linked glycosylation sites).

It may be desirable to modify an antibody with respect to effector function, for example to enhance antigen-dependent cell-mediated cytotoxicity (ADCC) and/or complement dependent cytotoxicity (CDC) of the antibody. This may be achieved by introducing one or more amino acid substitutions in an Fc region of the antibody. Alternatively, an antibody can be engineered which has dual Fc regions and may thereby have enhanced complement lysis and ADCC capabilities. See G. T. Stevenson et al., Anticancer Drug Des. 3:219-230 (1989).

To increase the serum half life of an antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

Various techniques have been developed for the production of whole antibodies and antibody fragments. Traditionally, antibody fragments were derived via proteolytic digestion of intact antibodies (see, e.g., K. Morimoto and K. Inouye, J. Biochem. Biophys. Methods 24:107-117 (1992); M. Brennan et al., Science 229:81-83 (1985)). However, these fragments can now be produced directly by recombinant host cells. Fab, Fv, V_{H}, V_{L}, and scFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (P. Carter et al., Biotechnology 10:163-167 (1992)). According to another approach, F(ab′)₂ fragments can be isolated directly from recombinant host cell culture.

A variety of expression vector/host systems may be utilized to express antibodies. These systems include but are not limited to microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems.

Expression vectors and host cells suitable for expression of recombinant antibodies and humanized antibodies in particular, are well known in the art. The following references are representative of methods and vectors suitable for expression of recombinant immunoglobulins which may be utilized in carrying out the present invention: Weidle et al., Gene, 51: 21-29 (1987); Dorai et al., J. Immunol., 13(12):4232-4241 (1987); De Waele et al., Eur. J. Biochem., 176:287-295 (1988); Colcher et al., Cancer Res., 49:1738-1745 (1989); Wood et al., J. Immunol., 145(9):3011-3016 (1990); Bulens et al., Eur. J. Biochem., 195:235-242 (1991); Beldsington et al., Biol. Technology, 10:169 (1992); King et al., Biochem. J., 281:317-323 (1992); Page et al., Biol. Technology, 9:64 (1991); King et al., Biochem. J., 290:723-729 (1993); Chaudhary et al., Nature, 339:394-397 (1989); Jones et al., Nature, 321:522-525 (1986); Morrison and Oi, Adv. Immunol., 44:65-92 (1989); Benhar et al., Proc. Natl. Acad. Sci. USA, 91:12051-12055 (1994); Singer et al., J. Immunol., 150:2844-2857 (1993); Couto et al., Hybridoma, 13(3):215-219 (1994); Queen et al., Proc. Natl. Acad. Sci. USA, 86:10029-10033 (1989); Caron et al., Cancer Res., 52:6761-6767 (1992); Coloura et al, J. Immunol. Meth., 152:89-109 (1992). Moreover, vectors suitable for expression of recombinant antibodies are commercially available. The vector may, for example, be a bare nucleic acid segment, a carrier-associated nucleic acid segment, a nucleoprotein, a plasmid, a virus, a viroid, or a transposable element.

Host cells known to be capable of expressing functional immunoglobulins include, for example: mammalian cells such as Chinese Hamster Ovary (CHO) cells; bacteria such as Escherichia coli; yeast cells such as Saccharomyces cerevisiae; and other host cells. Mammalian cells that are useful in recombinant antibody expression include but are not limited to VERO cells, HeLa cells, CHO cell lines (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), COS cells (such as COS-7), W138, BHK, HepG2, 3T3, RIN, MDCK, A549, PC12, K562, and 293 cells; myeloma cells, such as NS0 and SP2/0 cells as well as hybridoma cell lines. Mammalian cells are preferred for preparation of those antibodies that are typically glycosylated and require proper refolding for activity. Preferred mammalian cells include CHO cells, hybridoma cells, and myeloid cells. Of these, CHO cells are used by many researchers given their ability to effectively express and secrete immunoglobulins. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

In the production and use of antibodies, screening for or testing with the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme, or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, and the like.

Oligonucleotides Targeted to LL-37 and hCAP18

An oligonucleotide in a composition for therapeutic use may have a structure designed to achieve a well-known mechanism of activity including but not limited to a dsRNA-mediated interference (siRNA or RNAi), a catalytic RNA (ribozyme), a catalytic DNA, an aptazyme or aptamer-binding ribozyme, a regulatable ribozyme, a catalytic oligonucleotide, a nucleozyme, a DNAzyme, a RNA enzyme, a minizyme, a leadzyme, an oligozyme, or an antisense oligonucleotide. The oligonucleotides contemplated in this disclosure are targeted to pDC activation associated sequences, such as DNA encoding LL-37 precursor, hCAP18, and TLR-9, RNA (including pre-mRNA and mRNA) transcribed from such DNA, and also cDNA derived from such RNA. The pDC activation associated sequences may be any portion of the nucleic acid sequence, for example, an intragenic site or portion of an open reading frame (ORF), the 5′ untranslated region (5′UTR), the 5′ cap of an mRNA, which includes the first 50 nucleotides adjacent to the cap, and the like.

The term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars, and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target, and increased stability in the presence of nucleases. Thus, an oligonucleotide targeting a pDC activation associated sequence may be a DNA or a RNA molecule, or any modification or combination thereof. An oligonucleotide targeting an pDC activation associated sequence may contain, internucleotide linkages other than phosphodiester bonds, such as phosphorothioate, methylphosphonate, methylphosphodiester, phosphorodithioate, phosphoramidate, phosphotriester, or phosphate ester linkages (Uhlman et al., Chem. Rev. 1990; 90(4):544-584; Tidd, Anticancer Res. 1990; 10(5A):1169-1182), resulting in increased stability. Oligonucleotide stability may also be increased by incorporating 3′-deoxythymidine or 2′-substituted nucleotides (substituted with, e.g., alkyl groups) into the oligonucleotides during synthesis or by providing the oligonucleotides as phenylisourea derivatives, or by having other molecules, such as aminoacridine or poly-lysine, linked to the 3′ ends of the oligonucleotides (see, e.g., Tidd, 1990, supra). Modifications of the RNA and/or DNA nucleotides comprising the oligonucleotide targeting pDC activation associated sequence may be present throughout the oligonucleotide or in selected regions of the oligonucleotide, for example, the 5′ and/or 3′ ends. The oligonucleotide targeting pDC activation associated sequences can be made by any method known in the art, including standard chemical synthesis, ligation of constituent oligonucleotides, and transcription of DNA encoding the oligonucleotides. For example, the oligonucleotides may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. The oligonucleotides also may be produced by expression of all or a part of the target sequence in an appropriate vector.

In one embodiment, the oligonucleotide targeting a pDC activation-associated sequence may be an antisense oligonucleotide sequence. The antisense sequence is complementary to at least a portion of the 5′ untranslated, 3′ untranslated, or coding sequence. An oligonucleotide sequence corresponding to the agent targeting a pDC activation associated sequence must be of sufficient length to specifically interact (hybridize) with the target pDC activation associated sequence but not so long that the oligonucleotide is unable to discriminate a single based difference. For example, for specificity the oligonucleotide is at least six nucleotides in length. Longer sequences can also be used, depending on efficiency of inhibition, specificity, including absence of cross-reactivity, and the like. The maximum length of the sequence will depend on maintaining its hybridization specificity, which depends in turn on the G-C content of the agent, melting temperature (Tm) and other factors, and can be readily determined by calculation or experiment, for example, stringent conditions for detecting hybridization of nucleic acid molecules as set forth in “Current Protocols in Molecular Biology,” Volume I, Ausubel et al., eds. John Wiley:New York N.Y., pp. 2.10.1-2.10.16, first published in 1989 but with annual updating) or by utilization of free software such as Osprey (Nucleic Acids Research 32(17):e133) or EMBOSS (http://www.uk.embnet.org/Software/EMBOSS).

In another embodiment, the oligonucleotide may be an inhibitory RNA sequence (RNAi or siRNA) based on pDC activation associated sequences. Design of inhibitory RNA molecules is well known in the art and established parameters for their design have been published (Elbashir, et al. EMBO J. 2001; 20: 6877-6888). And methods of using RNAi-directed gene silencing are known and routinely practiced in the art, including those described in D. M. Dykxhoorn, et al., Nature Reviews 4:457-67 (2003) and J. Soutschek, et al., Nature 432:173-78 (2004). For example a target sequence beginning with two AA dinucleotide sequences are preferred because siRNAs with 3′ overhanging UU dinucleotides are the most effective. It is recommended in siRNA design that G residues be avoided in the overhang because of the potential for the siRNA to be cleaved by RNase at single-stranded G residues. The siRNA designed on the basis of a target pDC activation associated sequence can be produced by methods, such as chemical synthesis, in vitro transcription, siRNA expression vectors, and PCR expression cassettes. Irrespective of which method one uses, the first critical step in designing a siRNA is to choose the siRNA target site. Since a target sequence including flanking nucleotides is available for each pDC activation associated sequence, design of a suitable siRNA molecule is well within the knowledge of a skilled practitioner. Oligonucleotide targeting agents which recognize small variations of a core pDC activation associated sequence target are provided for in the present invention. The design of a suitable family siRNA molecule encompassing variant flanking sequences is well within the knowledge of a skilled practitioner. Thus, with knowledge of the target pDC activation associated sequence, the present invention provides for the design, synthesis, and therapeutic use of suitable siRNA molecules with will target pDC activation associated sequences.

In another embodiment, the oligonucleotide may be a ribozyme based on pDC activation associated sequences. Design and testing efficacy of ribozymes is well known in the art (Tanaka et al., Biosci Biotechnol Biochem. 2001; 65:1636-1644). It is known that a hammerhead ribozyme requires a 5′ UH 3′ sequence (where H can be A, C, or U) in the target RNA, a hairpin ribozyme requires a 5′ RYNGUC 3′ sequence (where R can be G or A; Y can be C or U; N represents any base), and the DNA-enzyme requires a 5′ RY 3′ sequence (where R can be G or A; Y can be C or U). Based on the foregoing design parameters and knowledge of the pDC activation associated sequence, a skilled practitioner will be able to design an effective ribozyme either in hammerhead, hairpin, or DNAzyme format. For testing the comparative activity of a given ribozyme, an RNA substrate which contains the common target sequence, i.e., an RNA containing a pDC activation associated, is used. Thus, with knowledge of the target pDC activation associated sequence, the present invention provides for the design, synthesis, and therapeutic use of suitable ribozymes which target pDC activation associated sequences in cells.

In another embodiment, the oligonucleotide may is an immunoregulatory sequences (IRS) that specifically inhibits TLR-9. These IRS sequences are ODN sequences on a phosphothiorate backbone (to protect from extracellular degradation.) These sequences are capable of binding to TLR-9, but fail to induce activation and may deliver inhibitory signals. U.S. Pat. No. 6,225,292, describes such inhibitors of TLR-9 suitable for use with the methods of the present disclosure.

Assay Systems

Any cell assay system that allows for assessing the function of pDC is contemplated by the present disclosure. The assay may be used to screen for compounds that inhibit or prevent production of pathogenic interferons. For example, such assays may be used to identify compounds that interact with LL-37, hCAP18, and TLR-9, which can be evaluated by assessing the effects of a test compound on the production of pathogenic interferons by pDC.

Typically, immunoassays use either a labeled antibody or a labeled antigenic component (e.g., that competes with the antigen in the sample for binding to the antibody). Suitable labels include without limitation enzyme-based, fluorescent, chemiluminescent, radioactive, or dye molecules. Assays that amplify the signals from the probe are also known, such as, for example, those that utilize biotin and avidin, and enzyme-labeled immunoassays, such as ELISA assays.

The disclosure also provides methods for visualizing or localizing a LL-37/DNA complex in tissues and cells. In one embodiment, biopsied tissues may be examined for presence of a LL-37/DNA complex in pDC. In another embodiment, an antibody-linked targeting agent or compound including a detectable label may be used to visualize or localize LL-37/DNA complex in pDC. As used herein, the term “detectable label” refers to any molecule which can be administered in vivo and subsequently detected. Exemplary detectable labels include radiolabels and fluorescent molecules. Exemplary radionuclides include indium-111, technetium-99, carbon-11, and carbon-13. Fluorescent molecules include, without limitation, fluorescein, allophycocyanin, phycoerythrin, rhodamine, and Texas red.

Pharmaceutical Compositions and Methods of Administration

Another aspect of the invention provides pharmaceutical compositions of the antibodies described above. The antibodies of the present disclosure can be mixed with pharmaceutically-acceptable carriers, excipients, or diluents to form a pharmaceutical composition for administration to a cell or subject, either alone, or in combination with one or more other modalities of therapy.

A pharmaceutical composition is generally formulated to be compatible with its intended route of administration. Those skilled in the art will know that the choice of the pharmaceutical medium and the appropriate preparation of the composition will depend on the intended use and mode of administration. Examples of routes of administration include parenteral (e.g. intravenous, intramuscular, intramedullary, intradernal, subcutaneous), oral (e.g. inhalation, ingestion), intranasal, transdermal (e.g. topical), transmucosal, and rectal administration. Administration routes for the antibodies of the present disclosure may also include intrathecal, direct intraventricular and intraperitoneal delivery. The antibodies may be administered through any of the parenteral routes either by direct injection of the formulation or by infusion of a mixture of the antibody formulation with an infusion matrix such as normal saline, D5W, lactated Ringers solution or other commonly used infusion media.

The antibodies of the present disclosure may be administered using techniques well known to those in the art. Preferably, agents are formulated and administered systemically. Techniques for formulation and administration may be found in “Remington's Pharmaceutical Sciences,” 18^{th} Ed., 1990, Mack Publishing Co., Easton, Pa. For injection, the antibodies may be formulated in aqueous solutions, emulsions, or suspensions. The antibodies are preferably formulated in aqueous solutions containing physiologically compatible buffers such as citrate, acetate, histidine, or phosphate. Where necessary, such formulations may also contain various tonicity adjusting agents, solubilizing agents and/or stabilizing agents (e.g. salts such as sodium chloride or sugars such as sucrose, mannitol, and trehalose, or proteins such as albumin or amino acids such as glycine and histidine or surfactants such as polysorbates (Tweens) or cosolvents such as ethanol, polyethylene glycol, and propylene glycol.

The pharmaceutical composition may contain formulation materials for modifying, maintaining, or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids, chelating agents [such as ethylenediamine tetraacetic acid (EDTA)]; solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. See Remington's Pharmaceutical Sciences, 18^{th} Edition, A. R. Gennaro, ed., Mack Publishing Company, 1990.

When parenteral administration is contemplated, the therapeutic compositions may be in the form of a pyrogen-free, parenterally acceptable aqueous solution comprising an antibody in a pharmaceutically acceptable vehicle. One vehicle for parenteral injection is sterile distilled water in which an antibody is formulated as a sterile, isotonic solution.

In another aspect, pharmaceutical formulations suitable for parenteral administration may be formulated in aqueous solutions, preferably in physiologically compatible buffers such as Hanks' solution, Ringer's solution, or a physiologically buffered saline. Aqueous injection suspensions may contain substances that increase the viscosity of the suspension, such as sodium carboxymethyl cellulose, sorbitol, or dextran. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

The pharmaceutical composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration.

For purposes of therapy, an antibody compositions and a pharmaceutically acceptable carrier are administered to a patient in a therapeutically effective amount. A combination of an antibody composition and a pharmaceutically acceptable carrier is said to be administered in a “therapeutically effective amount” if the amount administered is physiologically significant. An agent is “physiologically significant” if its presence results in a detectable change in the physiology of a recipient patient. A targeted therapeutic agent is “therapeutically effective” if it delivers a higher proportion of the administered dose to the intended target than accretes at the target upon systemic administration of the equivalent untargeted agent.

Therapeutic Methods

The compositions of the present disclosure have a variety of in vitro and in vivo diagnostic and therapeutic utilities. For example, these molecules can be administered to cells in culture, for example, in vitro or ex vivo. Alternatively, they can be administered to a subject, for example, in vivo, to treat a variety of disorders in which pathogenic interferon production plays a role. As used herein, the term “subject” is intended to include both human and nonhuman animals. The term “nonhuman animals” includes all vertebrates, for example, mammals and non-mammals.

The antibodies or binding fragments contemplated by the present disclosure may be used without modification, relying on the binding of the antibodies or fragments to the receptors, ligands, or molecules in the pathway leading to pDC activation and production of pathogenic interferons, thereby inhibiting function of the cells. Alternatively, the aforementioned method may be carried out using the antibodies or binding fragments to which a cytotoxic agent is bound. Binding of the cytotoxic antibodies, or antibody binding fragments, to the pDC may inhibit function of these cells, thereby providing a means for treating autoimmune diseases and chronic inflammatory diseases.

Human antibodies of this disclosure can be initially tested for binding activity associated with therapeutic use in vitro. For example, compositions of the invention can be tested using Biacore and flow cytometric assays. Suitable methods for administering antibodies and compositions of the present invention are well known in the art. Suitable dosages also can be determined within the skill in the art and will depend on the age and weight of the subject and the particular drug used.

Adjuvants

Developing efficient and safe adjuvants for use in human vaccines remains a challenge and necessity. Past approaches have been largely empirical and used adjuvants such as aluminium or emulsions. However new advances in basic immunology have elucidated how early innate immune signals can shape subsequent adaptive responses which have led to the design and development of more specific and focused adjuvants. In particular, a number of synthetic ligands for Toll-like receptors are currently being developed and test as novel adjuvants in cancer vaccines or vaccines against infectious diseases.

The present disclosure also provides compositions and methods for TLR9 agonist CpG-mediated therapy. Such may be used in the prevention and therapy of infectious disease; enhancing vaccines, and directing adaptive immunity without vaccine. We have shown that LL-37 can enhance IFN-α production by CpG sequences. And CpG sequences are widely used as adjuvants for anti-microbial vaccines, anti-tumor vaccines, and to inhibit allergic diseases such as asthma. Accordingly, LL-37 may be used to enhance immunogenicity of CpG and to enhance immunogenicity of anti-microbial vaccines that contain DNA (e.g., live, inactivated, or killed microbes). Accordingly, the present disclosure provides compositions comprising LL-37 plus CpGs as an adjuvant. Such compositions may also comprise, in addition to LL-37/CpGs, anti-microbial vaccines, anti-tumor vaccines, or other suitable vaccines.

A number of CpG sequences have been shown to enhance immunogenicity of anti-viral vaccines including HBV (J Clin Immunol 2003. 2:693-702, Vaccine 2004. 23:515-622) and influenza (Vaccine 2004. 22:3136-3143). As a monotherapy, CpGs given by injection, inhalation, or even by oral administration can protect against a wide range of viral, bacterial, and even some parasitic pathogens, including lethal challenge with Category A agents or surrogates such as Bacillus anthracis, vaccinia virus, Francisella tularensis, and Ebola virus. CpGs may also promote antitumor immunity as an adjuvant in vaccines or as a monotherapy administered systemically (reviewed in J Clin Invest 2007. 117:1184-1194). Murine models of allergic asthma have demonstrated that local administration of CpGs into the lungs can efficiently suppress allergic Th2 inflammation by promoting Th1 responses. Clinical trials are currently testing the efficacy of CpG inhalation for the treatment of allergic asthma. In all these settings, LL-37, according to certain embodiments of the present invention, may further enhance the therapeutic efficiency of CpGs.

The present disclosure also provides methods for using LL-37 alone as an adjuvant to enhance the immunogenicity of DNA/RNA therapeutic agent preparations, such as anti-microbial or anti-tumor vaccine preparations. For example, methods for treating a patient comprising administering to the patient a vaccine preparation, the vaccine preparation comprising DNA and/or RNA and an adjuvant comprising LL-37.

In general, suitable anti-microbial vaccine preparations containing DNA/RNA comprise vaccines containing bacteria or viruses. Examples of such vaccines include, but are not limited to, diphteria, polio, hepatitis, HIV, meningococcus, pneumococcus, meningococcus, group B streptococcus, and hospital acquired infections.

Suitable anti-tumor vaccine preparations that provide DNA/RNA for LL-37 binding include, but are not limited to, whole cell tumor vaccines, in which tumor cells (autologous or allogeneic) have been rendered apoptotic (e.g. by irradiation) or necrotic (e.g. by freeze/thaw cycles). These dying tumor cells may be premixed with LL-37 ex-vivo and administered into patients as a vaccine.

The present disclosure also provides methods for using LL-37 as monotherapy that targets self-DNA/RNA released by dying cells in-vivo. Tumors are characterized by a high degree of spontaneous cell death, which may be further enhanced therapeutically e.g. by radiotherapy. Thus, systemic LL-37-administration may specifically target tumors due to increased levels of cell death in the tumor microenvironment compared to healthy tissues. This specificity is unique to LL-37 and cannot be achieved by synthetic TLR9/7 agonists currently used in the clinics (e.g. CpGs and imidazoquinolines). LL-37 may also be delivered locally to the lungs of asthma patients by inhalation. Here LL-37 may couple with self-DNA/RNA released by dying cells in the context of inflammation. The induction of type I IFNs may convert the pathogenic proallergic Th2 response into a Th1 dominated response.

The compositions of this disclosure also can be co-administered with other therapeutic agents.

To facilitate a better understanding of the present invention, the following examples of specific embodiments are given. In no way should the following examples be read to limit or define the entire scope of the invention.

### Examples

Materials

The synthetic peptide wt LL-37 (LLGDFFRKSKEKIGKEFKRIVQRIKDFLRNLVPRTES) (SEQ ID NO. 1) and the mutated form (LLGDFFAVSKEKIGAEFVRIVQAIKDFLRNLVPRTES) (SEQ ID NO. 2) were purchased from Innovagen (Lund, Sweden). For confocal microscopy, the wt-peptide was covalently attached via cysteine residues to the fluorophore Texas Red (TR-LL-37). TR-LL-37 was purchased from the same company (Innovagen). Phosphorotioate (PT) and phosphodiester (PD) CpG 2216 (CpGA, GGGGGACGATCGTCGGGGGG (SEQ ID NO. 3)), CpG 2006 (CpGB, TCGTCGTTTTGTCGTTTTGTCGTT (SEQ ID NO. 4)), and the control ODN non-CpG sequence (TCCTGCAGGTTAAGT (SEQ ID NO. 5)) were produced by Trilink (San Diego, Calif.). The human TLR-9 signaling inhibitor (IRS, TTTAGGGTTAGGGTTAGGGTTAGGG (SEQ ID NO. 6)), Imiquimod (R837) and FITC-labeled CpG 2006 were from Invivogen (San Diego, Calif.).

Human genomic skin DNA (huDNA) was provided by BioChain (Hayward, Calif.). For confocal microscopy and flow cytometry huDNA was labeled with TOTO-3 fluorophore or with Alexa Fluor488 (Molecular Probes, Carlsbad, Calif.) according to the standard protocol provide by the manufacturer.

Dextran-647 and FM 0911 were from Molecular Probes. Chloroquine, Pertussis Toxin (PTX) and Adenosin triphopshate (ATP) were obtained from Sigma-Aldrich (Saint Louis, Mo.). WKYMV-peptide (W) was provided by ANASPEC (San Jose, Calif.). KN-62 was from AG Scientific, Inc. (San Diego, Calif.). DNase I was from Boehringer Mannheim, Indianapolis, Ind.).

Isolation and Stimulation of Plasmacytoid Dendritic Cells

PDC from healthy donors were purified from freshly collected buffy coats. Briefly, PBMC were isolated by Ficoll-Hypaque density gradient centrifugation (GE Healthcare, Piscatway, N.J.) followed by positive sorting using anti-BDCA4-conjugated magnetic microbeads (Miltenyi Biotec, Auburn, Calif.). The recovered cells were stained with PE-Cy5-conjugated anti-CD4, APC-conjugated CD11c, and a cocktail of FITC-conjugated anti-CD3, anti-CD14, anti-CD16, anti-CD15, anti-CD20 and anti-CD56 (Lineage-FITC) (BD Pharmingen, San Diego, Calif.).The CD4+CD11c-Lin− (pDC precursors) were isolated by cell sorting. Purity was routinely >99%. PDC (5-10×10⁴/well) were cultured in 96-well round-bottom plates in RPMI 1640 (GIBCO, Carlsbad, Calif.) supplemented with 10% FCS (Atlanta Biologicals, Lawrenceville, Ga.). Where indicated, pDC were stimulated with CpGA (1 uM), CpGB (1 uM), R837 (10 ug/ml), IRS (4 uM), non-CpG sequence (4 uM), and different concentrations of LL-37 and of human genomic DNA. To prepare LL-37·DNA complexes, CpGB, huDNA, non-CpG and LL-37 were mixed by inversion and incubated for 30 min at room temperature before being added to the cells.

Detection of Cytokines

Supernatants samples were taken after 18-24 h after addition of the stimuli. Human IFN-α was measured using a human IFN-α ELISA kit (PBL Biomedical Laboratories, New Brunswick, N.J.) according to the company's instructions. IL-6 and TNF-α were detected using a kit for human IL6 and TNF-α (R&D Systems), respectively.

Flow Cytometry

PBL were stained with antibodies to CD4 (APC-Cy7), CD11c (APC) and an antibody cocktail to lineage markers (CD3, CD14, CD15, CD16, CD20, CD56; all were FITC). Human pDC were identified and sorted by positive staining to CD4 and negative to CD11c and lineage markers. For phenotypic analysis cultured pDC were stained with antibodies to CD80 (FITC), CD123 (APC) and CD86 (PE) (all BD Pharmingen). Flow cytometry data were acquired on a FACSCalibur (BD Biosciences).

Real-Time Quantitative PCR

Lesional skin specimens were obtained from patients with psoriasis, lupus erythematosus (LE), prurigo nodularis (PN) and from healthy donors. Total RNA from homogenized skin was extracted with RNeasy kit mini protocol (Qiagen Inc., Valencia, Calif.) and was converted to cDNA using oligo-dT, random examers, and SuperScript II RT (Invitrogen, Carlsbad, Calif.). Quantitative Real-time polymerase chain reaction (PCR) was performed on a 7500 Fast Real-Time PCR System (Applied Biosystem, Foster City, Calif.) and target mixes (Applied Biosystem):

Confocal Microscopy

Confocal images were acquired using Leica SP2 RS SE scanner and sequential scanning with the 488 nm line of Ar laser and the 633 nm line of HeNe laser. Dual or triple color images were acquired by consecutive scanning with only one laser line active per scan to avoid cross-excitation.

Immunohistochemistry

Cryopreserved skin specimens were fixed in acetone, subsequently stained with an excess of primary Ab, including anti-human BDCA-2 mAb (Miltenyi Biotec) or anti-human LL-37 (HyCult Biotechnology). All sections were stained according to the indirect peroxidase method by using a Vectastain ABC Elite Kit (Vector Laboratories) and following the manufacturer's instructions.

Determination of AMP Involvement in pDC Activation

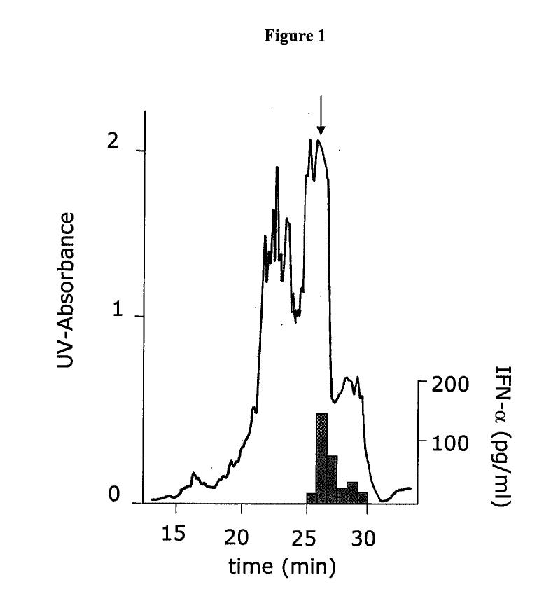

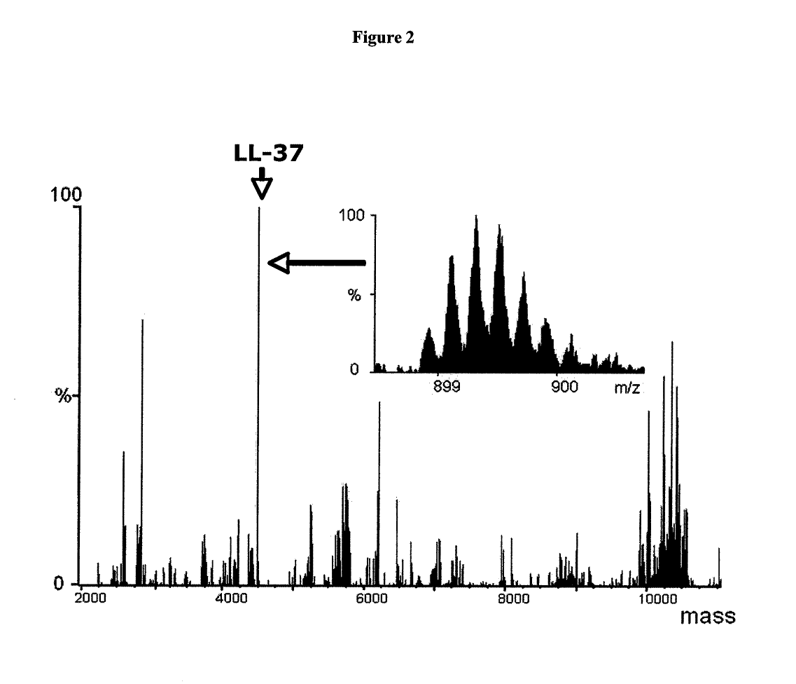

To search for a factor that specifically triggers pDCs to produce IFNs in psoriasis, we stimulated peripheral blood pDCs with extracts of psoriatic and healthy skin separated into fractions by preparative reversed-phase HPLC23. Whereas extracts of healthy skin were unable to activate pDCs (not shown), psoriatic extracts contained a major IFN-α-inducing fraction, which eluted after 26 min (FIG. 1). Using electrospray-ionization mass spectrometry (ESIMS) we identified two principal components of this fraction (Fraction 26): a 11,366 Da peptide and a 4,493 Da peptide. The 11,366 Da peptide was psoriasin, as previously reported, and the 4,493 Da peptide corresponded to the antimicrobial peptide LL-37, as confirmed by sequence analysis after nano-ESI-MS/MS of LysC digests (FIG. 2).

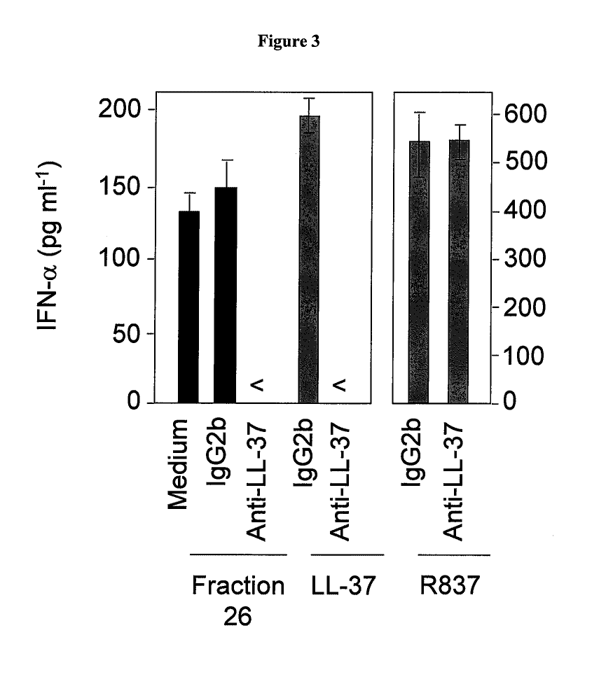

To investigate whether AMPS are involved in the activation of pDC to produce IFN-α, two sets of experiments were performed. In the first set, pDCs were stimulated with Fraction 26, LL-37 (3.9 μM) or R837 in the presence of anti-LL-37 (clone 8A8.2, produced by the methods described herein) or control antibodies (IgG2b). The results of this experiment are shown in FIG. 3. These data indicate that strategies that block the ability of LL-37 to bind self nucleic acids could be developed to prevent and/or treat psoriasis.

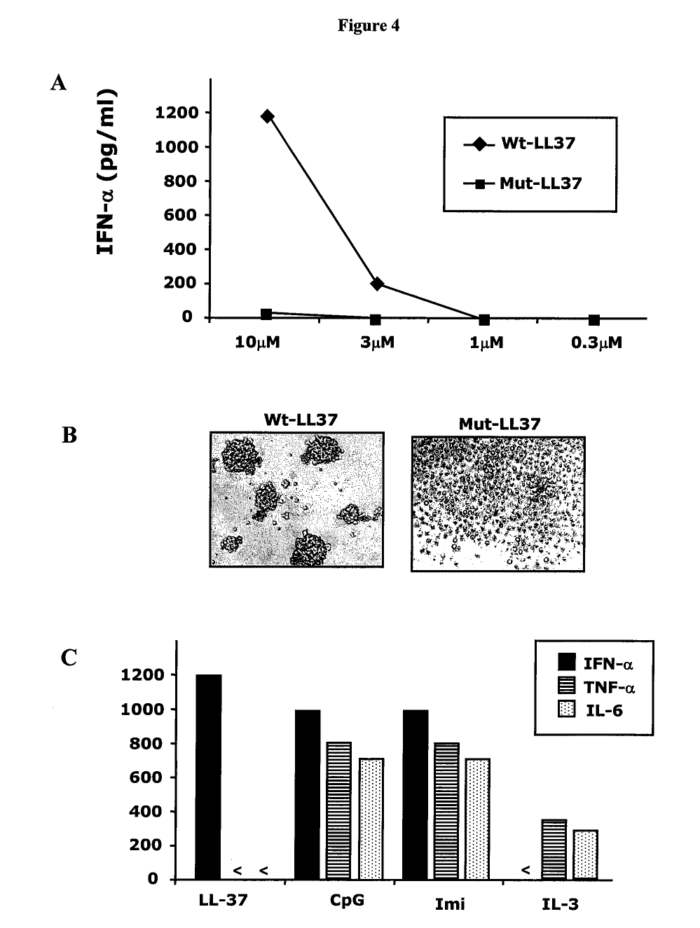

pDC were purified from human peripheral blood and cultured with equimolar doses of HBD-2, HBD-3, S100-7 and LL-37. Whereas non-stimulated pDC or pDC stimulated by HBD-2, HBD-3 or S100-7 did not induce pDC activation to produce IFN-α, cationic peptide LL-37 induced pDC to form clumps and produce significant levels of IFN-α (mean 950 pg/ml, range 200-4000, n=10) (FIGS. 4A and B). By contrast, stimulation with a mutated version of LL-37, called mLL-37, resulted in the complete abrogation of pDC activation (FIG. 4A). Interestingly, the capacity of LL-37 to activate pDC was seen in the presence of 10% serum in the culture medium, which was previously shown to abrogate the anti-microbial activity of LL-37.

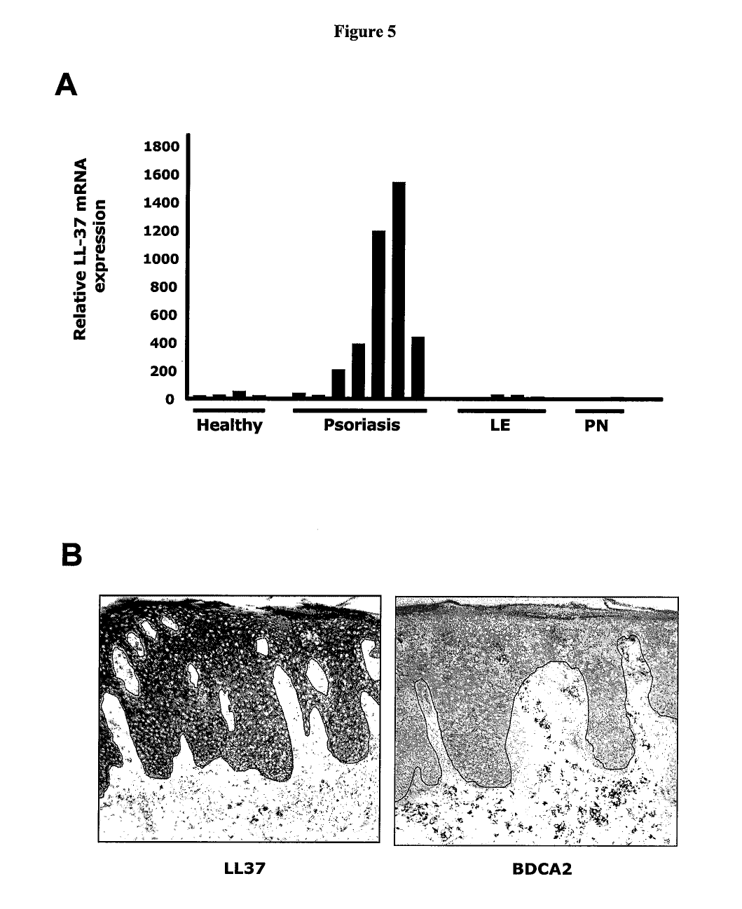

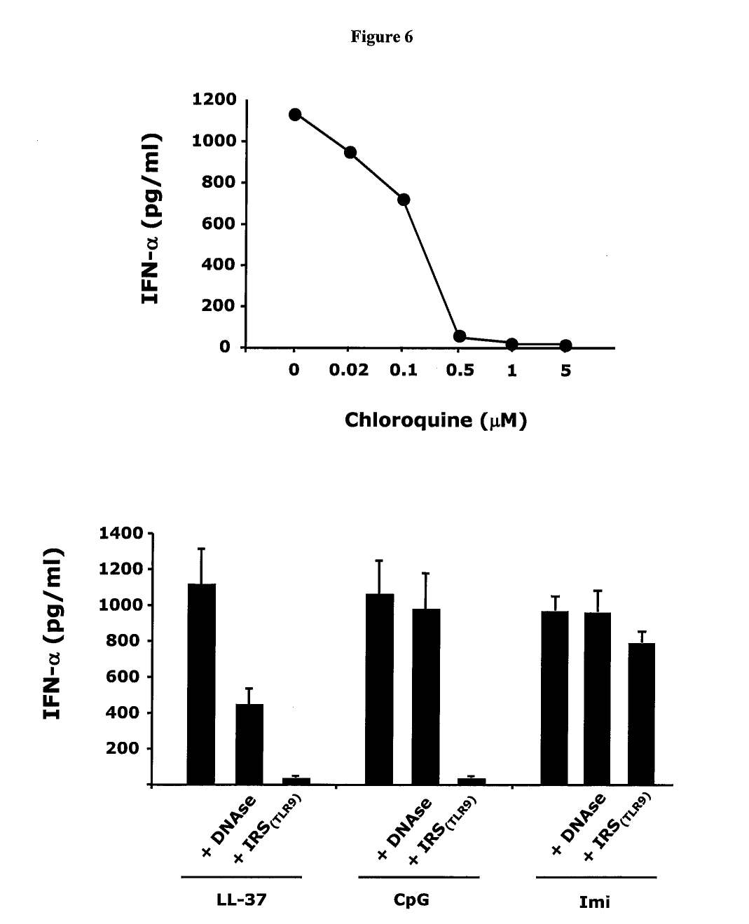

The levels of IFN-α induced by LL-37 were similar to those induced by TLR7 agonist imiquimod and TLR-9 agonist CpG-B (FIG. 4C). However, in contrast to imiquimod and CpG-B, LL-37 only induced IFN-α but not IL-6 or TNF-α (FIG. 4C) and did not induce maturation of pDC (not shown). LL-37 is a 37-residue cationic alpha-helical peptide and the only human member of the cathelicidine family of anti-microbial peptides. LL-37 expression in keratinocytes is inducible and rapidly upregulated after injury. LL-37 was highly expressed in inflammatory lesions of psoriasis but not in normal skin or skin lesions of Th1-inflammatory diseases such as LE and prurigo nodularis (FIG. 5A). Immunohistochemistry of psoriasis lesions revealed a strong epidermal expression of LL-37 and a significant subepidermal infiltration of pDC (FIG. 5B). LL-37 has direct anti-microbial effects on a broad range of bacteria, fungi and viruses. Furthermore LL-37 is involved in chemotaxis of mast cells, neutrophils and CD4 T cells via formyl peptide receptor-like 1 (FPRL-1), which belong to the Gi protein-coupled receptor family. Other host cell activities such as angiogenesis appear to be FPRL-1 independent and involve activation of P2X7. Thus next it was investigated whether the induction of IFN-α was mediated via FPRL-1 or P2X7. Blocking of the FPRL-1 and P2X pathway in pDC by inhibitors PTX and KN62, respectively did not inhibit IFN-α induction by LL-37 (FIG. 6A). Furthermore triggering these pathways by agonistic W peptide and ATP respectively did not result in IFN-α production by pDC (FIG. 6A). Given that the unique ability of pDC to secrete large amounts of IFN-α is based on recognition of microbial nucleic acids by endosomal TLR7 and TLR-9 we tested whether chloroquine, an inhibitor of endosomal acidification required for TLR7 or TLR-9 activation, abrogated the ability of LL-37 to induce IFN-α. Chloroquine inhibited LL-37-mediated IFN-α induction in a dose-dependent manner (FIG. 6B). The inhibition was not due to drug toxicity, because chloroquine had no measurable effect on on pDC viability (not shown). Thus activation of pDC to produce IFN-α appears to be independent of classical LL-37 receptors FPRL-1 and P2X and may involve endosomal TLR recognition.

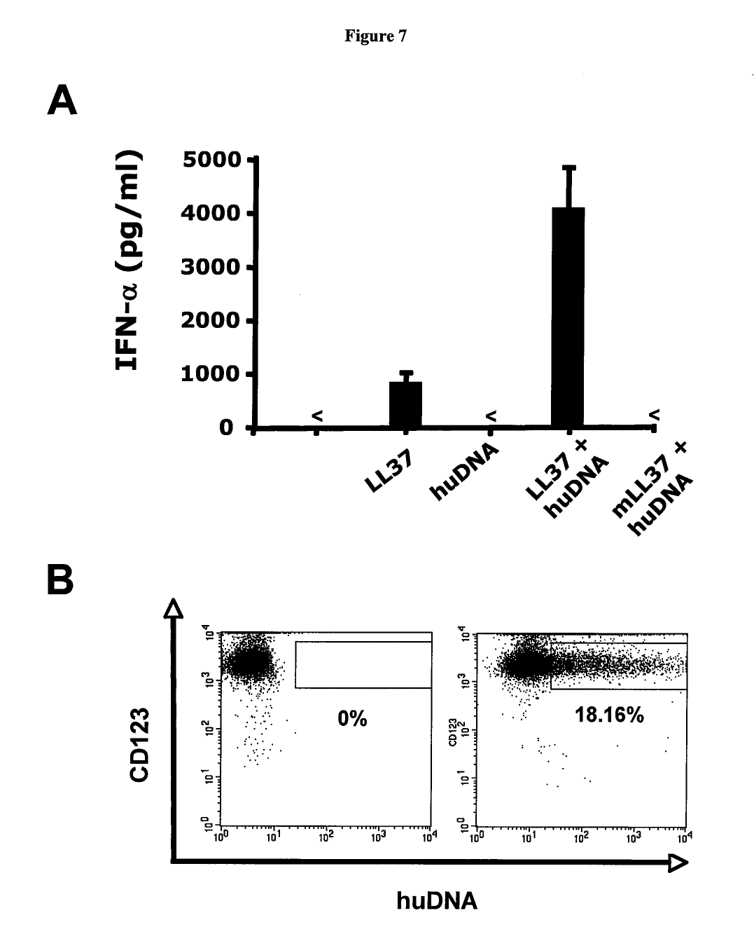

Given that LL-37 as a cationic peptide is unlikely to directly bind endosomal TLR which are receptors for negatively charged nucleic acids and given that cationic peptides with an alpha-helical structure like LL-37 can directly bind DNA, we hypothesized that LL-37 may bind DNA to activate endosomal TLRs. Addition of DNAse to the cultures significantly inhibited the LL-37-mediated activation of pDC to produce IFN-α (FIG. 6C). Specific blocking of TLR-9 by preincubation of pDC with immuno-regulatory ODN sequences (IRS) also inhibited pDC activation to produce IFN-α (FIG. 6C). The specificity of the IRS sequence for TLR-9 was shown by the ability to block IFN-α induction by TLR-9 agonist CpG-sequences but not TLR7 agonist imiquimod (FIG. 6C). Thus, LL-37 mediated activation of pDC to produce IFN-α occurs through TLR-9 and may involve DNA released into the cultures. To prove that LL-37 interacts with DNA to stimulate pDC, we cultured pDC with total genomic DNA either with or without pre-incubation with LL-37. Whereas genomic DNA alone was unable to activate pDC to produce IFN-α, genomic DNA plus LL-37 induced high levels of IFN-α (FIG. 7A).

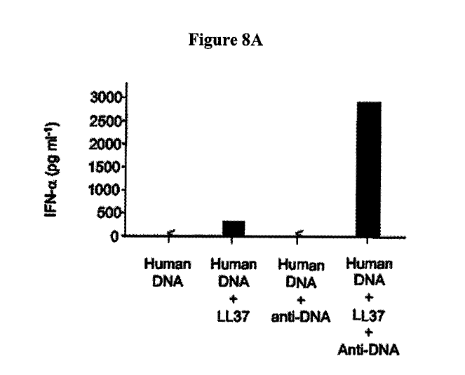

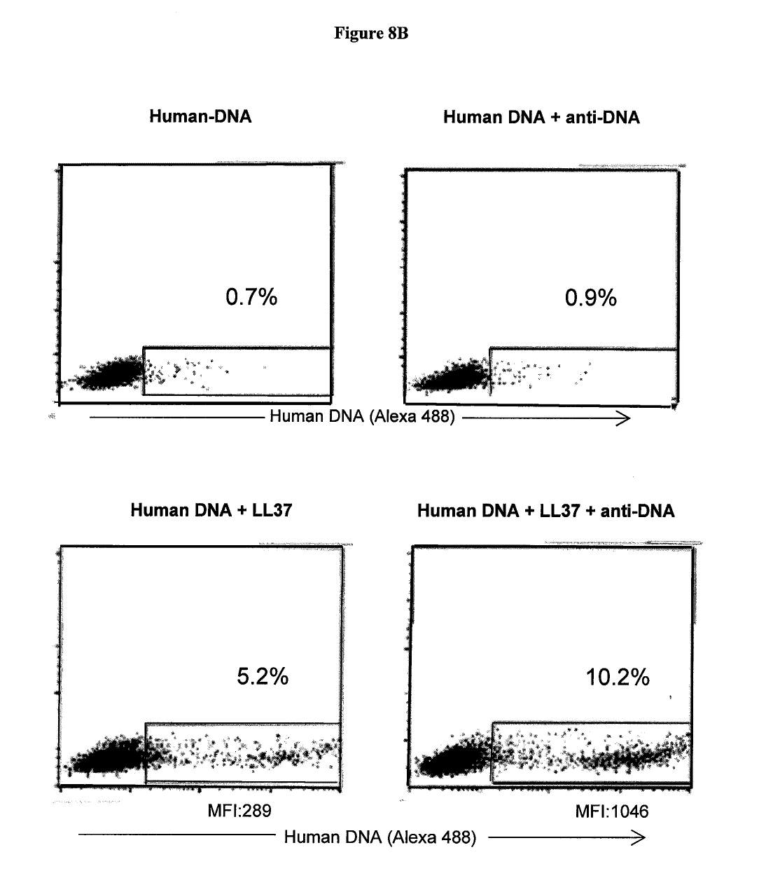

In accordance with these findings, flow cytometry analysis using fluorochrome-labeled genomic DNA revealed that, while DNA alone did not associate with pDCs (FIG. 7B, left panel), DNA pre-incubated with LL-37 readily associated with pDCs (FIG. 7B, right panel). Similarly, anti-DNA antibodies mixed with purified human genomic DNA are not sufficient to activate pDC to produce type I IFNs unless LL-37 is present. The antibody can however augment pDC activation by increasing the uptake of LL-37/DNA complexes (FIG. 8B). Indeed we found that LL-37 was present in immune complexes of SLE. Indeed purified total IgG from SLE sera contained LL-37 (FIG. 9, left panel) and depletion of LL-37-containing immune complexes abrogated the ability to induce IFN in pDC (FIG. 9, right panel). Together these data indicate that LL-37 and not antibodies are responsible for the break of innate tolerance to self-nucleic acids in SLE.

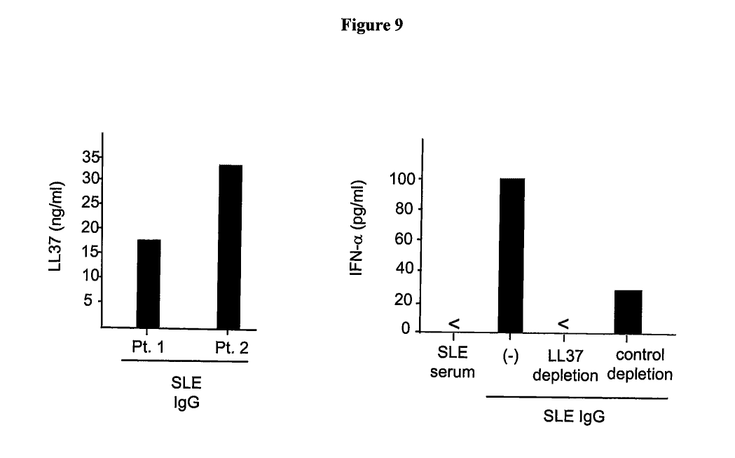

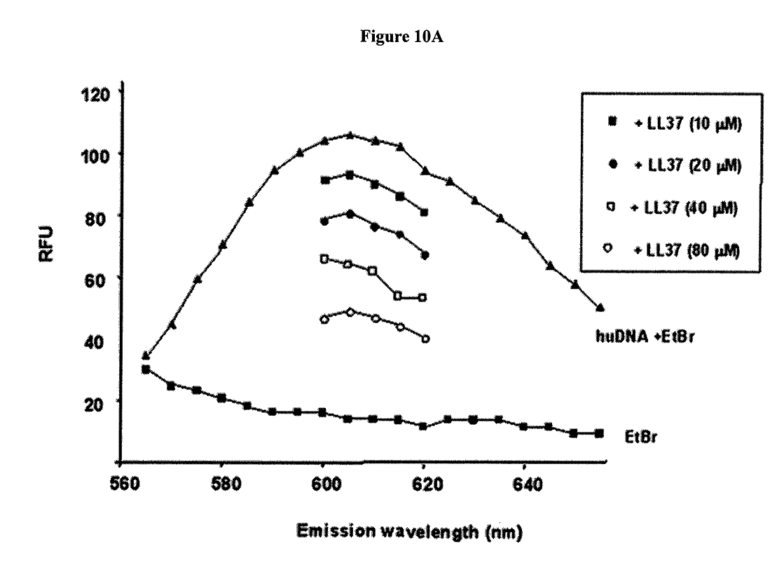

LL-37 complexed with DNA as shown by the ability of LL-37 to inhibit DNA intercalation (FIG. 10A), and by HPLC (FIG. 10B). By contrast, a mutated LL-37 peptide, in which the cationic residues had been substituted with neutral residues, was not able to complex with DNA (FIG. 10B), and accordingly did not induce IFN-α (FIG. 7A), indicating that the positive charges of LL-37 is of key importance in interaction with the DNA. We therefore sought to neutralize the positive charges of LL-37 by preincubation with heparin, a negatively charged protein. Indeed the ability of LL-37 to induce IFN-α was completely abrogated (FIG. 11).

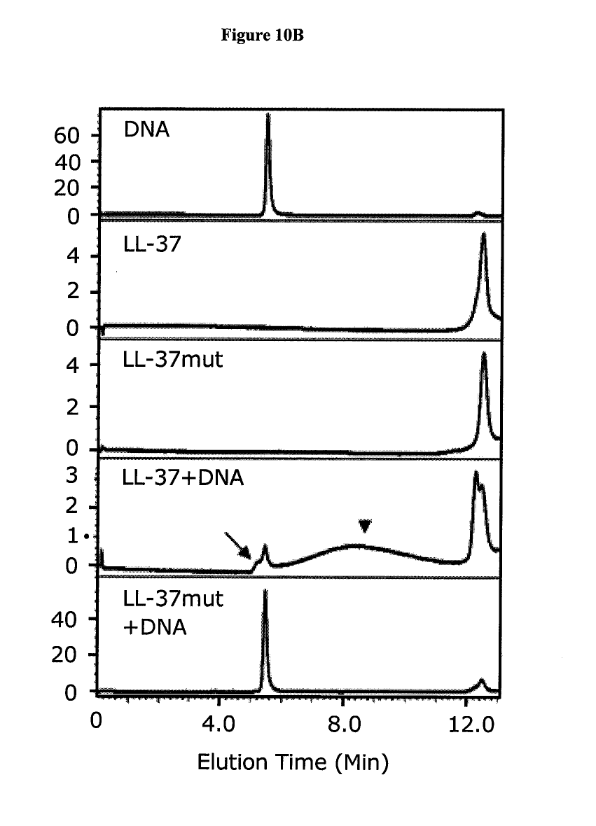

To determine the subcellular localization of the LL-37/DNA complex, pDC stimulated with the LL-37/DNA complex were stained with a membrane fluorescent marker and living cells were immediately examined by confocal microscopy. We observed the LL-37/DNA complex in small vesicular structures in the cell periphery at early timepoints (FIG. 12, 30 min after stimulation), moving towards the center of the cell at later timepoints (FIG. 12, 4 h after stimulation), Thus the complexed DNA/LL-37 is internalized to an endocytic compartment where it triggers TLR-9.

Recently, insight into the mechanism of TLR-9 triggering by short CpG-ODN sequences has been gained. LL-37 was also able to promote the IFN-α production of pDC in response to CpG-ODN, giving us the opportunity to analyze the mechanism of TLR-9 triggering by LL-37.

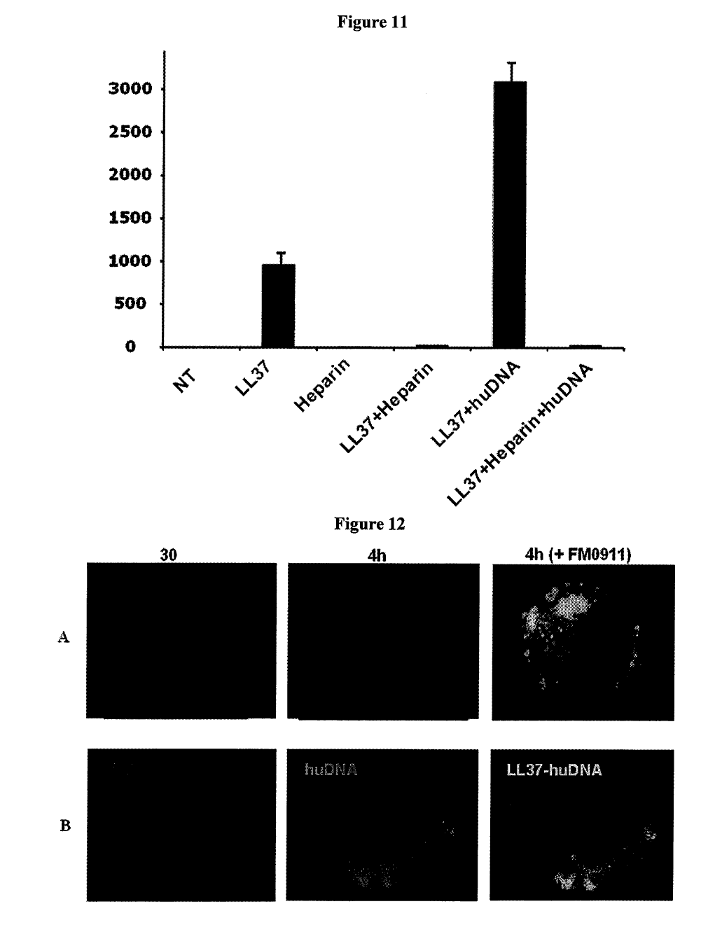

CpG-B sequences are synthetized with a phosphothiorate backbone to protect them from extracellular degradation. Indeed while phophodiesteric CpG-B was unable to induce IFN-α, phosphothiorate CpG-B induced significant levels of IFN-α (FIG. 13A). Addition of LL-37 to both phosphodiesteric and phosphothiorate sequences was able to induce large amounts IFN-α by pDC (approximately 10-fold more than induced by phophothiorate CpG-B alone) (FIG. 8A). These data indicate that LL-37 can indeed protect DNA from extracellular degradation but suggests additional mechanism to promote high levels of IFN-α. Interestingly, LL-37 was also able to induce significant levels of IFN-α in pDC stimulated with non-CpG-ODN sequences suggesting that the ability of LL-37 to promote DNA-mediated IFN-α induction may not be linked to specific DNA sequences (FIG. 13B). The ability of CpGA to induce huge amounts of IFN-α compared to CpG-B sequences depends upon their ability to form multimeric structures. Indeed the ability of CpGA to induce huge levels of IFN-α was strongly inhibited if the multimeric complex was disrupted and rendered single stranded by heat and flash cooling. However the potent interferogenic ability of CpGA was restored when complexed to LL-37 suggesting a role of LL-37 in forming multimeric structures with DNA (FIG. 7B). The ability of CpG sequences to induce large amounts of IFN-α by pDC has also been linked to the retention of CpG sequences in the early endomosome with consequent prolonged TLR-9 signalling. Indeed CpG-B complexed with synthetic cationic liposomes form aggregates that are retained for prolonged periods in early endosomes leading to enhanced IFN-α production by pDC. Similarly, whereas 2 h after pDC stimulation CpG-B alone was preferentially found in late endosomes (FIG. 13C, upper panel), CpG-B complexed with LL-37 colocalized in early endosomes at this timepoint (FIG. 13C, lower panel). Thus the effects of LL-37 on DNA appear to be a combination of extracellular protection from degradation, aggregate formation and retention in the early endosomes.

Blocking of LL-37 Cleavage from Propeptide by Proteinase 3 Inhibitors

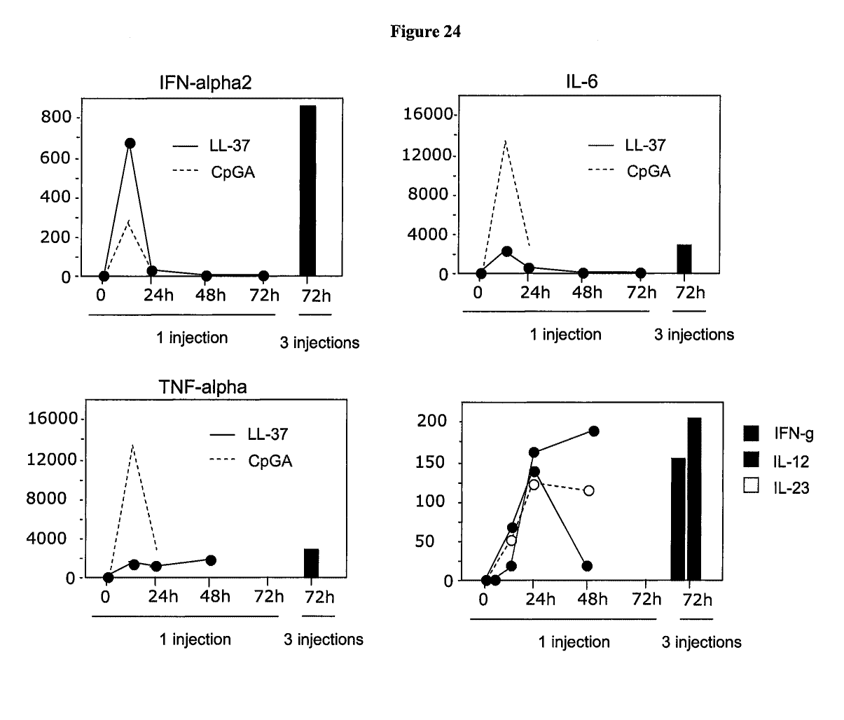

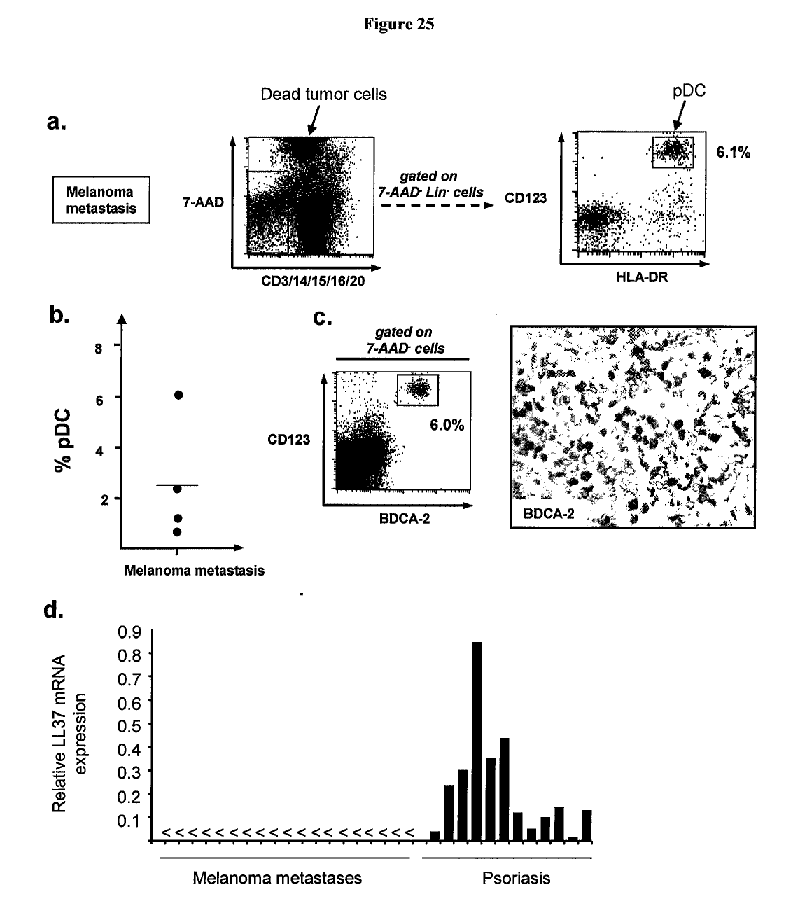

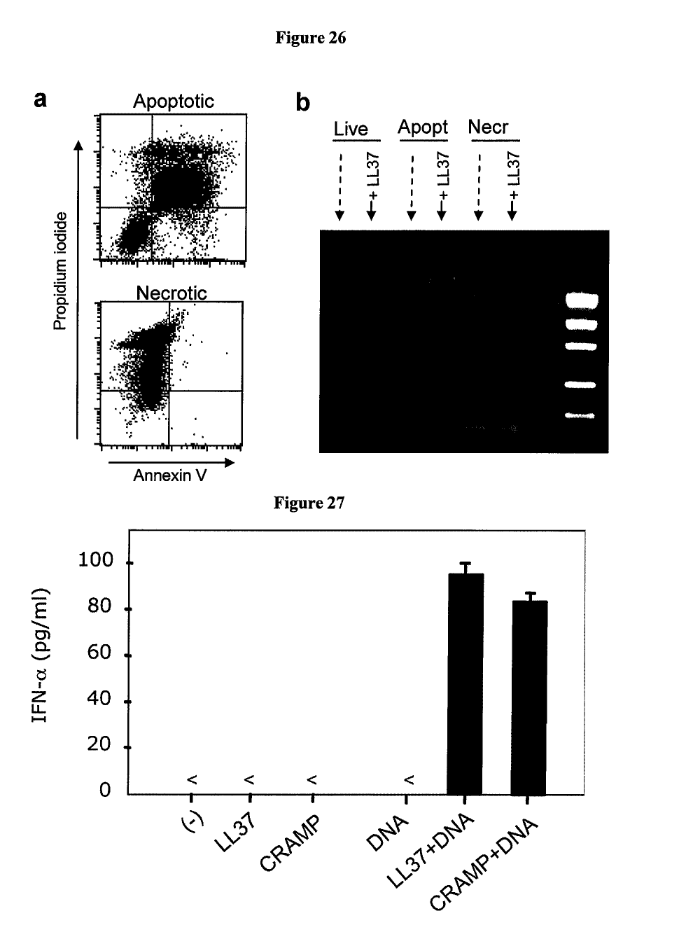

We now demonstrate in an in-vitro model of LL-37-DNA complex formation that blocking of LL-37 cleavage from pro-peptide hCAP18 inhibits type I IFN production by pDCs. We found that upon activation neutrophils release large amounts of self-DNA along with LL-37 (FIG. 25). We also found that these LL-37/self-DNA complexes activate pDC to produce type I IFNs (FIG. 26). Because the cleavage of the mature 4 kD LL-37 peptide from its inactive pro-peptide called hCAP18 requires proteinase 3 (Sorensen et al. Blood 2001, 97:3951) we used specific proteinase 3 inhibitors (Chymostatin or MeOSuc-CMK) to inhibit the generation of the active LL-37 peptide. FIG. 27 shows that the cleavage of the 4 kD LL-37 peptide can be blocked by pretreatment of neutrophils with the proteinase 3 inhibitors, and that the capacity of activated neutrophils to stimulate pDC to produce type I IFNs is abrogated. These findings indicate that proteinase 3 inhibitors block the generation of the mature LL-37 peptide, thus inhibiting the LL-37-mediated break of innate tolerance to self-nucleic acids.

Method for Generating Monoclonal Antibodies

a) Footpad Immunization. Antigen should be injected at 10 microgram per foot into a female BALB/c mouse. Immunizations will be done 6 times, at 3 days intervals.

b) Preparation of myeloma cells: P3-8AG-X653, or SP 2/0, grown in RPMI-1640 10% FBS. Cultures should be started at least two weeks before the projected fusion date. Always split the cultures in half the day before fusion.

c) Fusion. Three days after the sixth immunization the mouse is sacrificed and the popliteal lymph nodes removed. Using fine forceps and dissecting scissors, tease the nodes apart into 5 ml of serum-free RPMI-1640 media in a 60 mm dish. Transfer to a 15 ml conical tube, rinsing the dish with 5 ml addition S.F. media. Allow the larger chunks of tissue to settle while you harvest the myelomas. Carefully pipet up the suspended lymph node cells and transfer to a 50 ml conical tube. Lymph node cells and myeloma are washed twice in pre-warmed S.F. RPMI. Warm up 1 ml vial 30% PEG 1450, 5% DMSO, 65% S.F. RPMI, and a tube with 2 ml S.F. RPMI. Count the lymph node cells and myeloma; mix cells at a ratio of 3 lymph node: 5 myeloma. Centrifuge the mixed cells at 800 rpm for 7 min. Aspirate the supernatant and gently tap the tube to loosen the cell pellet. With a 1 ml pipet, add the PEG over 1 min. stirring with the pipet tip. Then stir the suspension for 1 min. with the pipet to thoroughly coat all the cells with PEG. With the same pipet, add 1 ml warm S.F. RPMI over 1 min. while stirring, then add another 1 ml S.F. RPMI over 1 min. while stirring. Then add 10 ml warm S.F. RPMI over 1 min. while stiring. Immediately centrifuge at 800 rpm for 7 min. Aspirate supernatant and tap the tube to loosen the pellet: avoid pipetting cells—PEG makes membranes fragile. Gently re-suspend cells in HAT medium: RPMI-1640, 10%FBS, 0.1 mM hypoxanthine, 0.4 uM aminopterin, 16 uM uM thymidine, add 10% rat spleen conditioned media. Distribute cells to sufficient 96-well plates to achieve cell concentration less than 5×10⁵ in 200 ul per well. I always include a control well of unfused myeloma cells, and usually a control well of unfused lymph node cells.

d) Feeding. On day 1, aspirate half of the media from each well and add 100 ul/well HAT media. Feed again on day 5, and every 2 days thereafter. I feed on a M/W/F schedule. By day 5, the unfused myeloma should be dying. Aminopterin can be omitted at this point. Hybridoma colonies should become visible within the week. The informal rule is colonies of at least ˜100 cells are required for sufficient signal to assay. This should take 10 days to 2 weeks. Supernatants will be assayed by ELISA. Briefly, the LL-37 peptide will be absorbed to the palate surface before the supernatants will be added and subsequently visualized by anti-mouse secondary antibodies. Positive wells should be transferred to 24-well plates, then frozen down and cloned out as soon as possible. Antibody fragments can be obtained using methods well-known in the art.

Method for Screening Inhibitory Activity of Generated mAbs In-Vitro

Human plasmacytoid DC will be purified from buffy coats of healthy donors. PBMC will be isolated by Ficoll-Hypaque density gradient centrifugation (GE Healthcare, Piscatway, N.J.) followed by positive sorting using anti-BDCA4-conjugated magnetic microbeads (Miltenyi Biotec, Auburn, Calif.). The recovered cells will be stained with PE-Cy5-conjugated anti-CD4, APC-conjugated CD11c, and a cocktail of FITC-conjugated anti-CD3, anti-CD14, anti-CD16, anti-CD15, anti-CD20 and anti-CD56 (Lineage-FITC) (BD Pharmingen, San Diego, Calif.).The CD4+CD11c-Lin- pDC precursors will be isolated by cell sorting. 5×10⁴/well pDC will be cultured in 96-well round-bottom plates in RPMI 1640 (GIBCO, Carlsbad, Calif.) supplemented with 10% FCS (Atlanta Biologicals, Lawrenceville, Ga.). The synthetic peptide LL-37 (Innovagen, Lund, Sweden) will be premixed at 100 m/ml with titrated concentrations of the generated anti-LL-37 mAbs in 100 μl of RPMI and incubated at room temperature for 30 minutes before adding 10 μg/ml genomic DNA extracted from human fetal skin (BioChain, Hayward, Calif.) and incubating at RT for additional 30 minutes. 5×10⁴/well pDC will be plated in 96-well round-bottom plates and the 100 ml of in RPMI 1640 (GIBCO, Carlsbad, Calif.) supplemented with 20% FCS (Atlanta biologicals, Lawrenceville, Ga.). After a total of 1 hour incubation, the 100 ml of the LL-37 mix (as described above) will be added to the same volume pDC cultures to yield a final concentration of 50 m/ml LL-37 in RPMI/10% FCS. pDC will be cultured for 24 h at 37C before supernatants are collected and assayed for IFN-α content by ELISA (PBL Biomedical Laboratories, New Brunswick, N.J.).

Method for Screening Inhibitory Activity of Generated mAbs In-Vivo

Purified mAbs generated with inhibitory activity in the in vitro assay described above will be tested in-vivo in a relevant model of human psoriasis. This is a xenotransplant model in which nearby uninvolved skin of a psoriatic patient is transplanted onto immunodeficient mice (RAG2^{-/-} combined with a common-γ chain^{-/-} or and AGR mouse) and currently represents the best preclinical psoriasis model. In this model the engrafted human skin converts spontaneously into a full-blown psoriatic plaque within 35 days of transplantation and is fully dependent on T cell activation. We have shown that this conversion is initiated by pDC activation to produce IFN-α at early stages after transplantation. pDC-derived IFN-α was necessary and sufficient to drive the activation of the autoimmune cascade leading to the development of psoriasis. Similar to our previous experiments using Abs against soluble molecules, we will inject 50 ug per mouse twice a week during the 5 weeks of psoriasis development.

Heparin Derivatives

Heparin, an anionic sugar which binds LL-37 through electrostatic interactions, has been used to inhibit the ability of LL-37 to complex will DNA and therefore inhibit activation of pDC to produce type I IFNs. Heparin derivatives can be engineered to retain binding to LL-37 but increasing safety profiles. For example, a heparin derivative may be a heparin-like molecule without the anticoagulatory properties.

Molecules Capable of Inhibiting TLR-9 Activation by the LL-37/DNA Complex

Activation of pDC to produce type I IFNs by the LL-37/DNA complex is mediated by endosomal toll-like receptor (TLR)-9. Activation of pDC to produce type I IFNs by the LL-37/RNA complex is mediated by endosomal toll-like receptor (TLR)-7. Thus specific inhibition of TLR-9/7 may block the activity of LL-37-DNA/RNA complex. Current strategies to specifically inhibit TLR-9/7 include the use of a class of oligonucleotides, named immunoregulatory sequences (IRS) described in issued U.S. Pat. No. 6,225,292. These IRS sequences are ODN sequences on a phosphothiorate backbone (to protect from extracellular degradation), which bind TLR-9/7 but fail to induce activation and may deliver inhibitory signals.

TLR-9 responses in pDC can be divided into two pathways; an early endosomal response mediated by IRF7 with consequent induction of type I IFN and a late endosomal response mediated by NFkB and dominated by the induction of TNF-α and induction of pDC maturation into DC. LL-37 has the ability to concentrate total DNA in early endosomes and specifically induce type I IFN and decrease maturation and TNF-alpha induction. CpG-A are a class of ODN with particularly effective induction of type I IFN by pDC due to their ability to form aggregates with consequent prolonged retention in early endosomal vescicles, Although IRS-ODN efficiently block low levels of type I IFN induction in pDC they fail to significantly suppress type I IFN induction by CpG-A.

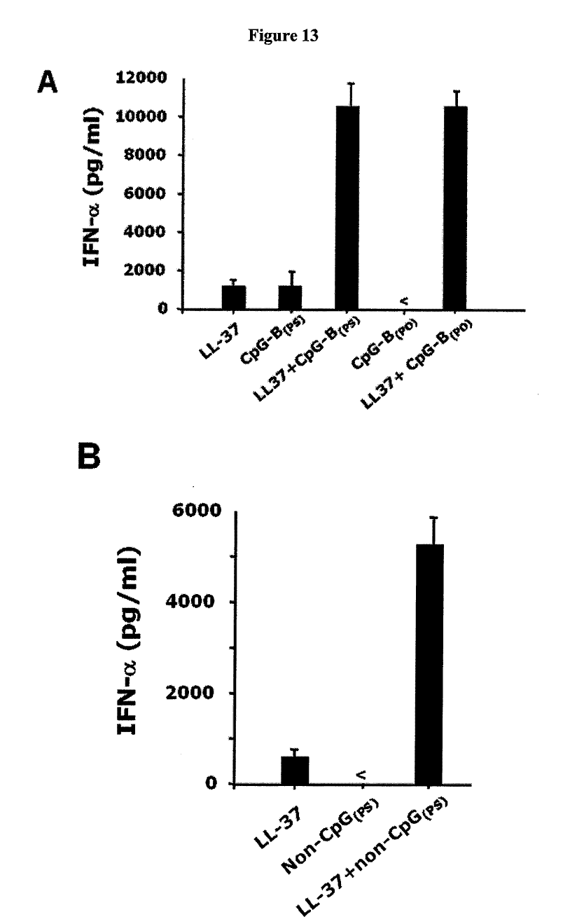

The ability of DNA to activate TLR-9 is best by sequences with multiple CpG. Indeed bacterial DNA, which contains multiple unmethylated CpG sequences strongly stimulate pDC activation through TLR-9. Although containing fewer such motifs also mammalian DNA can become a potent stimulator of TLR-9 when concentrated in the endosomes. It has been shown that that CpG motifs in both dsDNA and ssDNA sequences are required for the induction of type I IFN by the LL-37/DNA complex. In contrast LL-37 complexed with CpG-free DNA sequences is not able to induce type I IFN (FIG. 13). We also show that LL-37 complexed with a non CpG-containing ODN is able to completely (>90%) inhibit the activation of pDC by strong type I IFN inducers such as CpG-A (FIG. 14). The data indicate that LL-37 preincubated with a non-CpG ODN is able to strongly inhibit the activation of pDC by CpGA (FIG. 11).

Use of LL-37 as an Adjuvant in Human Vaccines

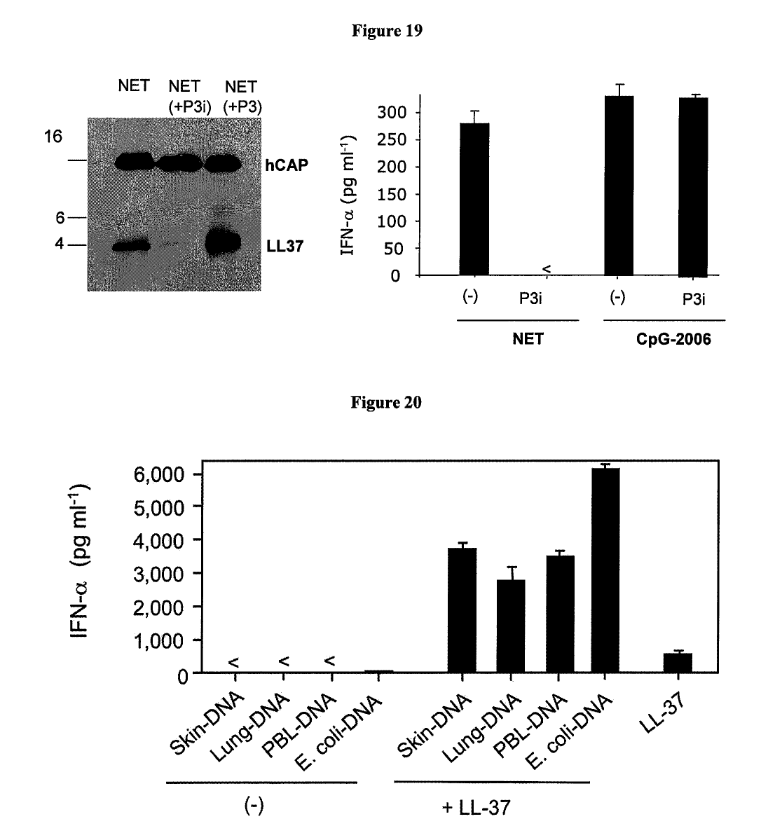

In order to investigate the potential use of LL-37 as an adjuvant in human vaccines, a series of experiments were performed. In the first set, pDCs were stimulated with genomic DNA derived from human fetal skin, human lungs and human leukocytes (10 μg ml⁻¹) either alone or after premixing with LL-37 (10 μM). pDCs were also stimulated with genomic bacterial DNA isolated from Escherichia coli (E. coli) at 10 μg ml⁻¹. Levels of IFN-α were measured after overnight culture. The results of this experiment are shown in FIG. 20. These results show that LL-37 converts genomic DNA of human and bacterial origin into potent IFN-α inducers.

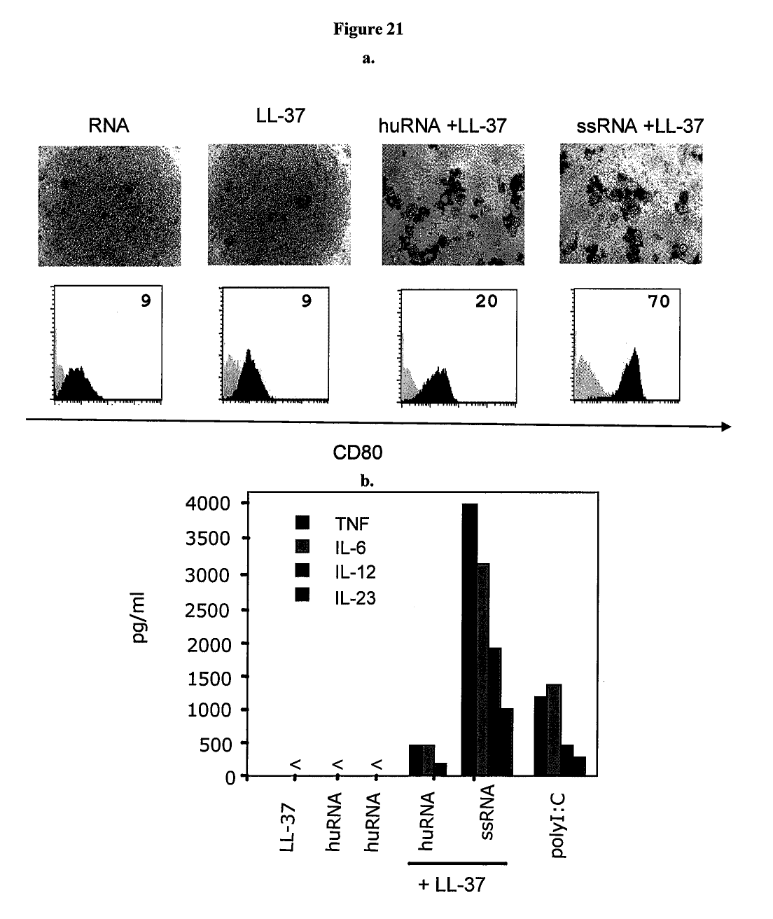

In a second set of experiments, myeloid (monocyte-derived) DC were stimulated with RNA isolated from U937 cells (human RNA) or a synthetic single-stranded RNA sequence derived from HIV (ssRNA40) and a known TLR-7/8 ligand either alone (10 μg ml⁻¹) or after premixing with LL-37 (10 μM). Maturation was assessed by flow cytometry analysis of CD80 after overnight culture (FIG. 21A). Levels of TNF-α, IL-6, IL-12, and IL-23 were measured after overnight culture (FIG. 21B). These results show that LL-37 converts self-RNA and viral RNA into activator of myeloid DC maturation and cytokine secretion.

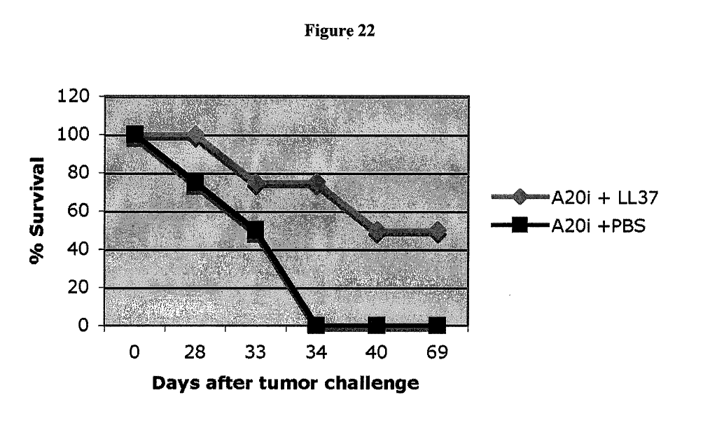

In a third set of experiments, 10⁶ A20 irradiated (5000 rad) were mixed with LL-37 (30 μg) or left in PBS alone and injected subcutaneously. 7 days later mice were challenged with live A20 lymphoma i.v. 8 mice per group, survival over time is plotted. The results of this vaccination experiment are shown in FIG. 22. These results show that vaccination with LL-37 plus dying tumor cells induces prolonged survival of tumor challenged mice.

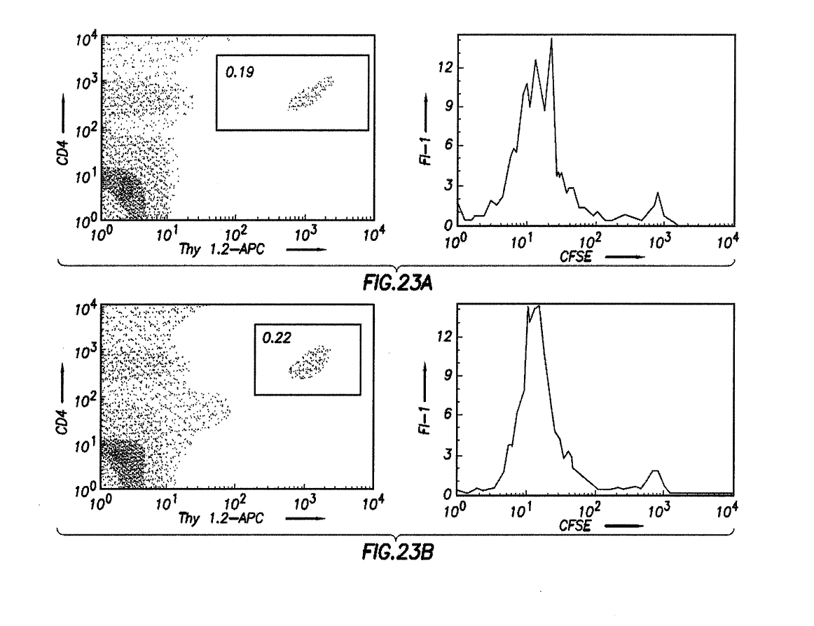

In another set of experiments, CD4+ T cells were purified from spleen and LN of HNT-TCR Tg mice (Thy 1.2), labeled with CFSE, and adoptively transferred (1×10⁶) into BALB/c Thy1.1 mice. Next day, mice were immunized subcutaneously with 5×10⁶ A20 lysate plus HNT peptide and CpG-2216 (35 μg), A20 lysate plus HNT peptide and LL-37 (35 μ), A20F lysate plus HNT peptide, or left untreated. Four days after immunization draining LN were harvested and Thy1.2 positive CD4 T cells were measured by flow cytometry. The results of this experiment are shown in FIG. 23. These results show the potent adjuvant activity of LL-37 for the induction of T cell mediated immunity.

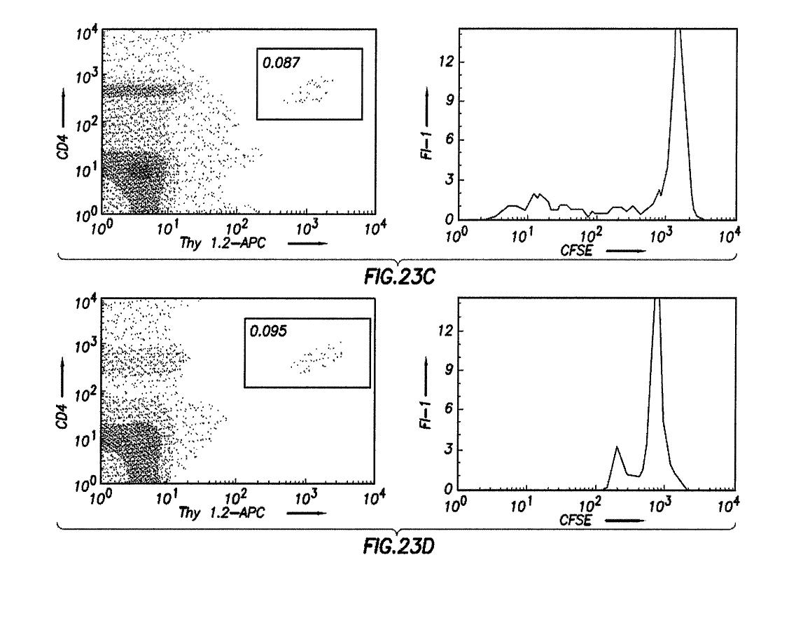

In another set of experiments, 100 μg of LL-37, CpG-A or PBS alone was injected into B16 tumors grown for 7 days in Flt-L treated mice. Tumors were harvested after 6, 24, 48 and 72 h, total RNA was extracted and expression of indicated cytokines was measured by real-time PCR. The data, shows in FIG. 24, represent expression relative to GAPDH RNA. Some mice were injected with 100 μg of LL-37 for 3 times (t0, t24 and t48) and tumor was harvested at 72 h for RNA expression analysis. These results show that intratumoral injection of LL-37 induces expression of pro-inflammatory and T-cell-derived cytokines.

Human melanoma tumor contains pDCs in the vicinity of dying tumor cells but does not express LL-37. Human blood pDC can be identified by their unique surface expression profile lacking common lineage markers for T, B, NK and monocytes and expressing CD123, HLA-DR and BDCA-2. In mononuclear cell suspensions generated from solid melanoma metastases, we found consistently high numbers of lineage⁻HLADR⁺CD123⁺ pDC (mean 2.7% of mononuclear cells) (FIG. 25a, b). As for blood pDCs, BDCA-2 appear to be specific for tumor pDC because the frequency of BDCA2+ cells was identical to the frequency of lineage⁻HLADR⁺CD123⁺ cells (FIG. 25c). Immunohistochemistry for BDCA-2 confirmed that substantial numbers of pDCs can infiltrate the tumor microenvironment of human melanoma metastases (FIG. 25c).

Flow cytometry revealed considerable amount of dead tumor cells, identified by the typical FSC/SSC scatter and by 7-AAD staining (FIG. 25a). The presence of dying tumor cells suggests the presence of self-DNA released into the extracellular compartment. Because pDC have the potential to be activated by self-DNA released by dying cells in the presence of LL-37 (FIG. 25c), we determined whether LL-37 is expressed in the melanoma tissue. By real-time PCR analysis, we found that LL-37 mRNA expression was completely absent in tissue of melanoma metastases (n=19) (FIG. 25d). These data suggest that human melanoma tumor metastases contain pDCs and self-DNA but lack LL-37. Providing LL-37 to the tumor may therefore convert self-DNA into a trigger of pDC leading to an anti-viral-like innate immune activation.

LL-37 combined with dying tumor cells can bind tumor-derived self-DNA in-vitro. To test the ability of LL-37 to bind and protect self-DNA released by dying tumor cells, we generated apoptotic and necrotic tumor cells in the presence or absence of LL-37, and measured DNA contents in culture supernatants by electrophoresis. Primary necrosis induced by consecutive freeze and thaw cycles, and apoptosis with secondary necrosis induced by γ-irradiation at 20,000 rad followed by a 24 h culture (at 5×10⁶ cells in 500 μl) were confirmed by Annexin plus PI staining (FIG. 26a). By electrophoresis we exclusively detected DNA in supernatants of irradiated tumor cells cultured with LL-37 (FIG. 26b). These results indicate that irradiated tumor cells release self-DNA that is bound and protected by LL-37. By measuring the fluorescence of DNA stained with a specific dye (Sytox Green at 523 nm) we found that concentration of DNA in our cultures was routinely >10 μg ml⁻¹ (determined in comparison to a standard curve using known concentrations of purified genomic DNA).

Murine pDC respond to LL-37-DNA complexes. To determine whether mouse pDCs can respond to LL-37/DNA complexes, we purified mouse pDCs from Flt3L-supplemented BM cultures according to their CD11c+CD11b−B220+ phenotype, and stimulated them with DNA complexed with LL-37 or CRAMP (the murine LL-37 homologue). We found that both LL-37/DNA and CRAMP/DNA were able to induce type I IFN production. However, compared to LL-37, approximately 3 times more CRAMP was required to elicit the same amount of type I IFNs (FIG. 27).

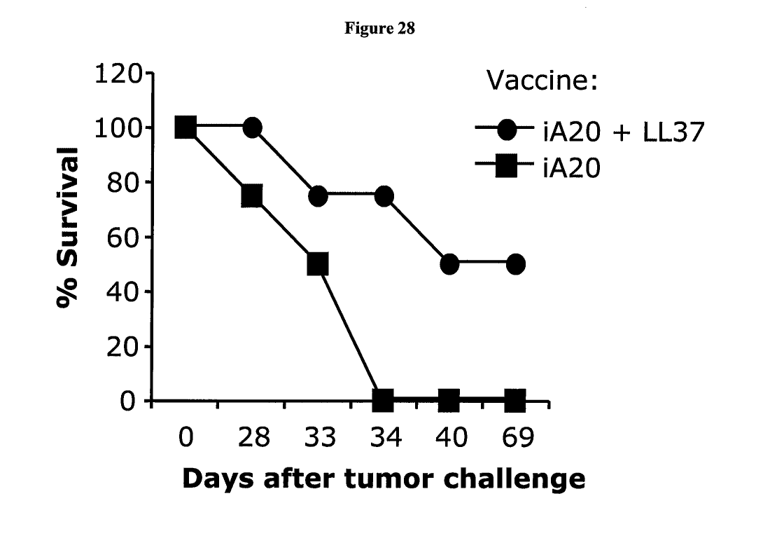

LL-37 combined with dying tumor cells and injected as a vaccine has potent anti-tumor activity. In a murine model of B-cell lymphoma called A20, BALB-c mice were inoculated intravenously with 10⁷ A20 lymphoma cells. The mice typically succumb after 5-7 weeks to disseminated lymphoma affecting lymph nodes, spleen and liver. We found that a single subcutaneous injection of LL-37 mixed with irradiated A20 tumor cells induced prolonged survival of mice inoculated with tumor cells 7 days later (FIG. 28). Whereas 5 weeks after inoculation all mice without treatment had succumbed, 80% of the vaccinated mice were still alive. This data suggest that this vaccination may limit the systemic spread of the inoculated lymphoma.

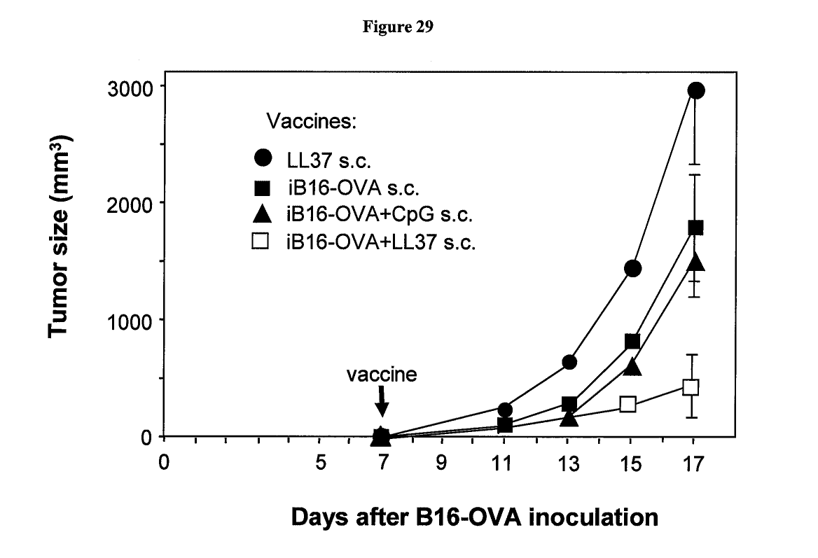

We also performed vaccine studies using the B16 tumor model of melanoma. B16 is a highly aggressive tumor with low immunogenicity. B16 tumor cells can be transfected with ovalbumin (OVA) to provide an immunogen that allows easy tracking of the anti-tumor immune response. 3×10⁵ B16-OVA tumor cells were implanted subcutaneously in the flank of C57BL/6 mice and allowed to grow. Seven days later mice were treated with a single subcutaneous injection of LL-37 mixed with irradiated B16-OVA tumor cells. Control injections included LL-37 alone, irradiated B16-OVA alone, or irradiated B16-OVA mixed with the synthetic TLR9 agonist CpG. A detailed method on the generation of these vaccines is provided in D2.1. Tumor size was monitored with a caliper and volumes estimated using the formula π/6×length×width. The experiment was stopped 10 days after injection because all mice in the control group had died or their tumor had reached 20 mm in their maximal diameter. Vaccination with LL-37 plus irradiated tumor cells significantly delayed the growth of 7-day established B16 tumor cells compared to the control groups and even irradiated B16-OVA mixed with CpGs (FIG. 29). Together these data indicate that LL-37 combined with dying tumor cells and injected as a vaccine shows potent antitumor activity, suggesting the induction of T cell-mediated anti-tumor immunity. LL-37 appears to be more potent than CpGs, among the most potent adjuvants currently tested in clinical vaccination trials. These experiments were done using CpG-2216, which is the most potent CpG-sequence for the ability to induce type I IFNs in pDCs.

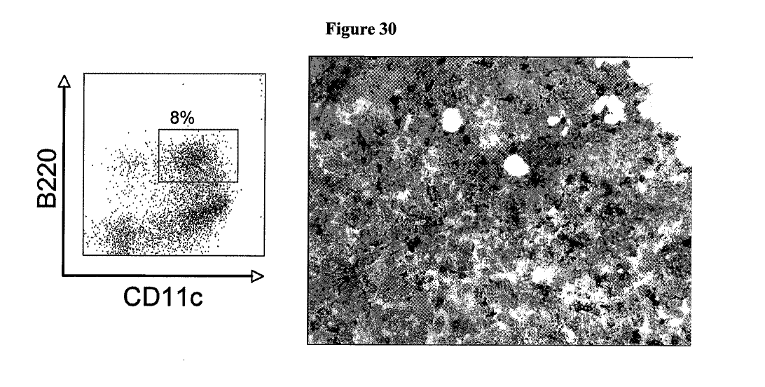

Murine B16 melanoma contains large numbers of pDC along with dying tumor cells. To confirm that murine B16 melanoma would model human melanoma and contain increased numbers of pDC and self DNA, C57BL/6 mice were left untreated or pretreated for 4 days with the expression vector encoding a full-length murine Flt3-ligand cDNA, using the hydrodynamic-based gene delivery technique. This procedure is a useful tool to expand DC populations in the tumor, thus facilitating the analysis of DC-specific events. After 4 days, 3×10⁵ B16 melanoma cells were inoculated into shaved flanks and allowed to grow for 7 days. At day 7 tumors were harvested and divided into 2 pieces. One piece (¼) was snap-frozen for immunohistochemical analysis of 3H3, a specific marker for murine pDC. The remaining part (¾) was used to generate single cell suspensions for flow cytometry analysis for B220⁺CD11c⁺ pDCs. B16 melanoma contained large numbers of pDCs as determined by flow cytometry and histology (FIG. 30). Whereas untreated mice have approximately 1-3% pDC in their tumors, Flt3-ligand treated mice have about 6-9% (FIG. 30). As described for human melanoma, murine B16 melanoma is characterized by extensive tumor cell death in the tumor microenvironment (FIG. 30), as well as the lack of LL-37 expression.

Intratumoral injection of LL-37 into native unmodified B16 melanoma induces potent type I IFN expression. 3×10⁵ native B16 melanoma cells were inoculated into C57BL/6 mice. After 7 days, tumors were injected with 100 μg of LL-37, 40 μg CpG-2216 (CpG-A), or saline (PBS). Because LL-37-DNA binding (which will occur in the tumor) is optimal at a 3:1-5:1 ratio, we injected approximately 3 times more LL-37 than CpG-DNA. Tumors were harvested at 6 h, 24 h, 48 h, and 72 h after injection and total RNA was isolated and processed. Expression of IFN-α2 mRNA was measured by real-time PCR and normalized for expression of GAPDH mRNA.

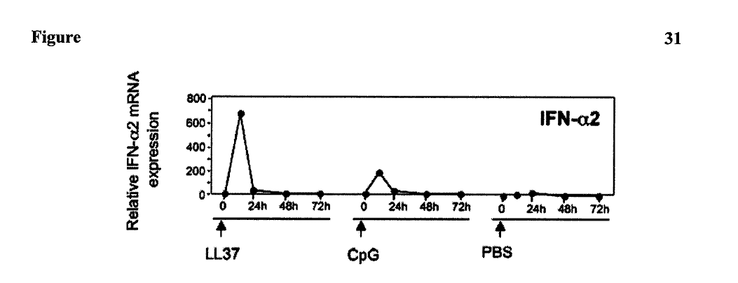

We found that intratumoral injection of LL-37 induced potent IFN-α2 mRNA expression (FIG. 31). Strikingly expression was more potent then the expression induced by CpG-A, the most potent CpG sequence for the ability to induce type I IFNs. These data indicate that intratumoral injection of LL-37 can induce an anti-viral-like innate immune response with expression of type I IFNs in the tumor microenvironment. LL-37 appears to be more potent than CpG for the ability to induce type I IFN expression in-vivo.

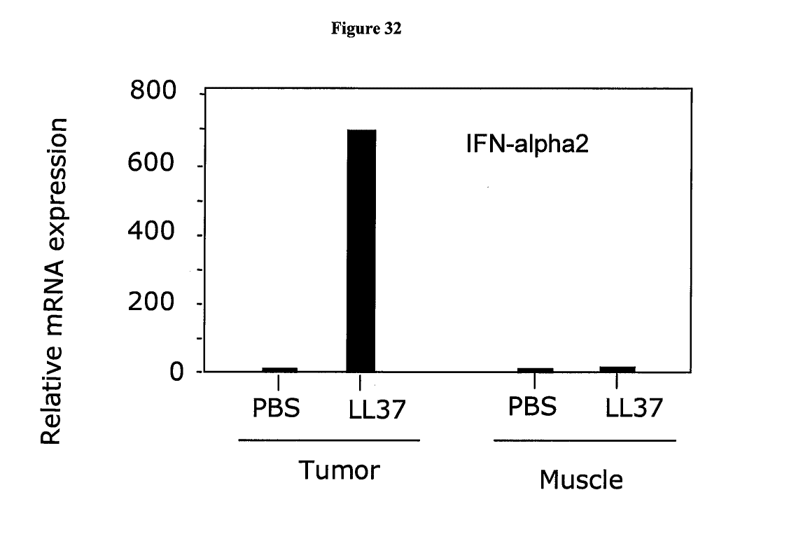

LL-37 can induce type I IFN expression when injected into tumors but not into healthy tissue. Because LL-37 requires the presence of self-DNA released by dying cells to induce pDC activation to produce type I IFN, we next asked whether LL-37 could selectively induce type I IFN expression in tumors (containing a high degree of cells death) and not in healthy tissue. To address this question we injected LL-37 (100 μg) into subcutaneously implanted B16 tumors as well as healthy muscle tissue. 6 h after injection, tissues were collected and IFN-α2 mRNA expression was measured by real-time PCR, as described in C12. We found IFN-α2 mRNA expression only in LL-37-injected tumors but not LL-37-injected healthy muscle tissue (FIG. 32). These data suggest that LL-37 targets dying cells to induce an anti-viral-like innate immune activation in the tumor while not affecting healthy tissues.

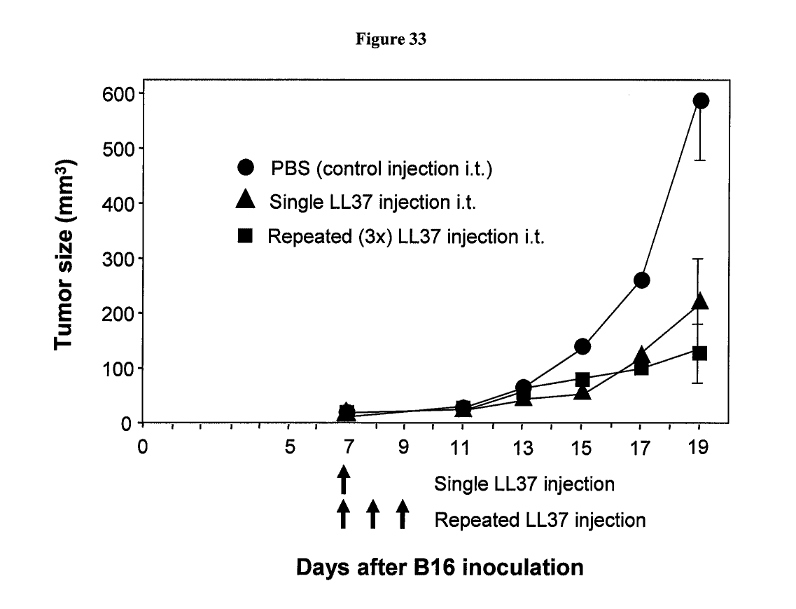

Intratumoral injection of LL-37 elicits potent anti-tumor activity. 3×10⁵ B16 tumor cells were inoculated into shaved flanks of C57BL/6 mice. Tumors were allowed to grow for 7 days. On day 7, tumors were either injected with a single dose of LL-37 (100 μg), injected daily for 3 consecutive days with LL-37 or injected with saline as a control. Tumor size was monitored with a caliper and volumes estimated using the formula π/6×length×width². The experiment was stopped 12 days after injection because all mice in the control group had died or their tumor had reached 20 mm in their maximal diameter. We found that a single intratumoral LL-37 injection significantly delayed the growth of established B16 tumor (FIG. 33). Repeated LL-37 injection on three consecutive days showed a trend towards a better anti-tumor response. Thus, intratumoral LL-37 injection induces potent anti-tumor activity.

The above studies demonstrated, among other things, that:
LL-37 has the unique ability to convert inert self-DNA released by dying cells into a potent trigger of pDC activation to produce type I IFNs. This occurs by binding self-DNA to form aggregated and condensed structures that are delivered to endocytic compartments in pDCs to trigger TLR9.
LL-37 is extraordinarily potent in driving type I IFN production due to its ability to concentrate and retain DNA in early endocytic compartments of pDC. This may explain why LL-37/DNA is more potent than synthetic CpG-DNA in its ability to induce type I IFNs in-vitro and in-vivo.
LL-37 combined with dying tumor cells can bind self-DNA released by the tumor cells.
LL-37 combined with dying tumor cells ex-vivo and injected as a vaccine can inhibit growth of established melanoma.
The tumor microenvironment of melanoma contains a high degree of dying cells, resulting in abundant extracellular self-DNA. It also contains large numbers of non-activated pDC but lacks LL-37 expression.
Direct intratumoral LL-37 injection induces potent type I IFN expression.
LL-37 specifically induces type I IFNs in tumors but not healthy tissue upon direct injection.
Direct intratumoral LL-37 injection can inhibit growth of established melanoma.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. An antibody comprising a human constant region that binds to a marker specific for pDC cell activation by host (self) nucleic acids that may lead to production of pathogenic interferons or for specific blocking of LL-37 induced immune reactivity to self nucleic acids leading to pathogenic type I IFNs.

2. The antibody of claim 1, wherein the marker specific to pathogenic interferon production is chosen from one or more of LL-37 and hCAP18.

3. The antibody of claim 1, which is chimeric.

4. The antibody of claim 1, which is humanized.

5. The antibody of claim 1, which is a human sequence antibody.

6. The antibody of claim 1, which is a monoclonal antibody.

7. A molecular inhibitor comprising an oligonucleotide that binds to a marker specific to pathogenic interferon production.

8. The inhibitor according to claim 7, wherein the inhibitor is an oligonucleotide which binds under high stringency conditions to a polynucleotide encoding one or more of LL-37 and hCAP18.

9. The inhibitor according to claim 8, wherein the oligonucleotide comprises non-naturally occurring bonds.

10. The inhibitor according to claim 8, wherein the oligonucleotide comprises an immunoregulatory sequence.

11. A molecular inhibitor comprising a small molecule that inhibits pathogenic interferon production.

12. The inhibitor according to claim 11, wherein the small molecule chosen from one or more of a heparin, a heparin derivative, and an anionic polymer.

13. A vector encoding and capable of expressing an oligonucleotide according to claim 7.

14. A method for inhibiting pathogenic interferon production or inhibiting activation of plasmacytoid dendritic cells or treating an autoimmune or chronic inflammatory disease, which comprises inhibiting one or more of LL-37 and hCAP18.

15. The method according to claim 14, wherein inhibiting one or more of LL-37 and hCAP18 comprises administering a proteinase 3 inhibitor.

16. The method according to claim 14, further comprising administering a therapeutically effective amount of at least one of (i) an antibody as defined in any of claims 1-6; (ii) a molecular inhibitor as defined in any of claims 7-12; or (iii) a vector as defined in claim 13.

17. The method according to claim 15, further comprising administering a vector encoding and capable of expressing an oligonucleotide according to claim 7 in a human cell.

18. A pharmaceutical composition comprising a therapeutically effective amount of at least one of (i) an antibody as defined in any of claims 1-6; (ii) a molecular inhibitor as defined in any of claims 7-12; or (iii) a vector as defined in claim 13.

19. A method for identifying a marker specific to pathogenic interferon production comprising screening a plurality of cells for one or more of LL-37 and hCAP18 and identifying the cells that express one or more of LL-37 and hCAP18.

20. A composition comprising an LL-37 and a CpG sequence.

21. A composition comprising an anti-microbial or an anti-tumor vaccine containing DNA and/or RNA and an LL-37.

22. A method for enhancing the immunogenicity of a DNA and/or RNA therapeutic agent preparation comprising providing to the preparation LL-37, and optionally a CpG sequence.

23. The method according to claim 22, wherein the preparation is an anti-microbial or anti-tumor vaccine.

24. The method according to claim 22, wherein the preparation is an anti-microbial vaccine derived from bacteria, viruses, or both.

25. The method according to claim 22, wherein the preparation is an anti-tumor vaccine derived autologous or allogenic tumor cells.

26. A method for treating a patient comprising administering to the patient a composition according to claim 21.


SEQUENCE LISTING (7 sequences)

SEQ ID NO: 1 - protein, 37 aa, Artificial Sequence
  Synthetically created peptide
LLGDFFRKSKEKIGKEFKRIVQRIKDFLRNLVPRTES

SEQ ID NO: 2 - protein, 37 aa, Artificial Sequence
  Mutated form of synthetically created peptide
LLGDFFAVSKEKIGAEFVRIVQAIKDFLRNLVPRTES

SEQ ID NO: 3 - DNA, 20 bp, Artificial Sequence
  Synthetically created CpG sequence
gggggacgatcgtcgggggg

SEQ ID NO: 4 - DNA, 24 bp, Artificial Sequence
  Synthetically created CpG sequence
tcgtcgttttgtcgttttgtcgtt

SEQ ID NO: 5 - DNA, 15 bp, Artificial Sequence
  Synthetically created non-CpG sequence
tcctgcaggttaagt

SEQ ID NO: 6 - DNA, 25 bp, Artificial Sequence
  Synthetically created sequence
tttagggttagggttagggttaggg

SEQ ID NO: 7 - protein, 12 aa, Artificial Sequence
  Fragment of synthetically created sequence
DFLRNLVPRTES